(19) United States
(12) Patent Application Publication
(10) Pub. No.: US 2016/0346289 A1
(43) Pub. Date: Dec. 1, 2016

(54) Fixed-Dose Combinations of Antiviral Compounds

(71) Applicants: Pavithra SUNDARARAJAN, West Point, PA (US); Agam SHETH, West Point, PA (US); Jesse KUIPER, West Point, PA (US); Patrick Jules MARSAC, West Point, PA (US); Elise MILLER, West Point, PA (US); Justin David MOSER, West Point, PA (US); Adam SOCIA, West Point, PA (US); Angela M. WAGNER, Austin, TX (US); MERCK SHARP & DOHME CORP., Rahway, NJ (US)

(72) Inventors: Pavithra Sundararajan, North Wales, PA (US); Agam Sheth, Plymouth Meeting, PA (US); Jesse Kuiper, North Wales, PA (US); Patrick Jules Marsac, Lexington, KY (US); Elise Miller, Gilbertsville, PA (US); Justin David Moser, Collegeville, PA (US); Adam Socia, Blue Bell, PA (US); Angela M. Wagner, Austin, TX (US)

(73) Assignee: Merck Sharp & Dohme Corp., Rahway, NJ (US)

(21) Appl. No.: 15/116,824

(22) PCT Filed: Feb. 3, 2015
(86) PCT No.: PCT/US15/14201

§ 371 Date: Aug. 4, 2016

Related U.S. Application Data
(60) Provisional application No. 61/936,019, filed on Feb. 5, 2014.
(60) Provisional application No. 62/095,427, filed on Dec. 22, 2014.
(60) Provisional application No. 62/095,398, filed on Dec. 22, 2014.

(51) Int. Cl. A61K 31/5365; A61K 9/28; A61K 9/20; A61K 9/00; A61K 31/4985; A61K 9/10

(52) U.S. Cl. CPC A61K 31/5365; A61K 31/4985; A61K 9/10; A61K 9/2027; A61K 9/2054; A61K 9/2013; A61K 9/0053; A61K 9/28; A61K 9/2893

(57) ABSTRACT

The present disclosure is directed to compositions comprising blended materials comprising a first solid dispersion formulation, which comprises an HCV NS3/NS4a protease inhibitor or a pharmaceutically acceptable salt thereof, one or more pharmaceutically acceptable polymers or a mixture thereof, and optionally one or more pharmaceutically acceptable surfactants or a mixture thereof; a second solid dispersion formulation, which comprises an HCV NS5a inhibitor or a pharmaceutically acceptable salt thereof, one or more pharmaceutically acceptable polymers or a mixture thereof; and optionally one or more pharmaceutically acceptable surfactants or a mixture thereof; and optionally one or more excipients. The present disclosure is also directed to oral dosage forms, such as tablets or capsules comprising the disclosed blended compositions comprising the disclosed solid dispersion formulations, and the methods for making these solid dispersion formulations and pharmaceutical compositions.

### FIELD OF THE INVENTION

The instant invention relates to pharmaceutical formulations that are useful for the treatment of diseases and disorders caused by hepatitis C virus (“HCV”). In particular, the pharmaceutical formulations are fixed-dose combinations that comprise solid dispersion formulations of two or more antiviral compounds.

### BACKGROUND OF THE INVENTION

Hepatitis C virus (HCV) infection is a major health problem that leads to chronic liver disease, such as cirrhosis and hepatocellular carcinoma, in a substantial number of infected individuals. Current treatments for HCV infection include immunotherapy with recombinant interferon-α alone or in combination with the nucleoside analog ribavirin. Potential treatments for HCV infection have been discussed in the different references including Balsano, 8(4) MINI REV. MED. CHEM. 307-318, 2008; Rönn et al., 8 CURRENT TOPICS IN MEDICINAL CHEMISTRY 533-562, 2008; Sheldon et al., 16(8) EXPERT OPIN. INVESTIG. DRUGS 1171-1181, 2007; and De Francesco et al., 58 ANTIVIRAL RESEARCH 1-16, 2003. Several virally encoded enzymes are putative targets for therapeutic intervention, including a metalloprotease (non-structural (NS) 2-3), a serine protease (NS3, amino acid residues 1-180), a helicase (NS3, full length), an NS3 protease cofactor (NS4A), a membrane protein (NS4B), a zinc metalloprotein (NS5A) and an RNA-dependent RNA polymerase (NS5B).

One potential avenue for treatment is combination therapy, in which two or more antiviral agents are co-administered, with each antiviral agent acting on one or more of these non-structural regions as therapeutic targets. The combination of two or more antiviral agents acting on different non-structural regions may provide a combination drug product having additive effects for viral load suppression. Indeed, the landscape for treatment of HCV is trending towards an all-oral, direct-acting antiviral regimen that is active against all HCV genotypes, and combination drug products that comprise two or more HCV antiviral agents, each acting on a different therapeutic targets, may form a crucial component of an all-oral regimen.

The NS3 protease is located in the N-terminal domain of the NS3 protein, and is considered a prime drug target because it is responsible for an intramolecular cleavage at the NS3/4A site and for downstream intermolecular processing at the NS4A/4B, NS4B/5A and NS5A/5B junctions. NS3/NS4 inhibitor compounds include compounds such as grazoprevir, (1aR,5S,8S,10R,22aR)—N-[(1R,2S)-1-[(cyclopropylsulfonamido)carbonyl]-2-ethenylcyclopropyl]-14-methoxy-5-(2-methylpropan-2-yl)-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide hydrate, which is shown below as Compound I:

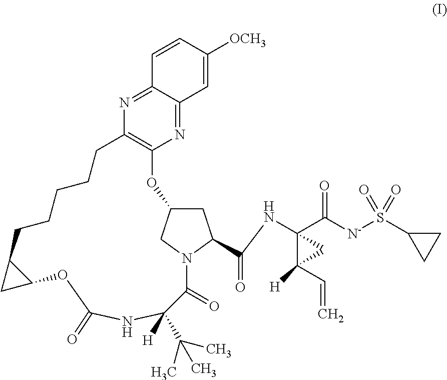 [SMILES: C=C[C@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)[N-]S(=O)(=O)C1CC1]

Compound I is described in U.S. Pat. No. 7,973,040. Compound I is a selective HCV NS3/NS4A inhibitor. It is a poorly water soluble, moderately lipophilic compound. These properties make conventional formulation approaches challenging. See generally, U.S. Provisional Patent Application No. 61/936,019, filed Feb. 5, 2014.

Another identified target for therapeutic intervention is the HCV NS5A non-structural protein, which is described, for example, in Seng-Lai Tan & Michael G. Katze, 284 VIROLOGY 1-12 (2001); and in Kyu-Jin Park et al., 278(33) J. BIO. CHEM. 30711 (2003). A non-structural protein, NS5A is an essential component for viral replication and assembly. Mutations in NS5A at or near known sites of phosphorylation can affect the ability for high-level replication in cell-culture systems, suggesting an important role for NS5A phosphorylation in viral replication efficiency Inhibitors of the phosphorylation of NS5A can lead to reduced viral RNA replication.

NS5A inhibitor compounds include compounds such as elbasvir (dimethyl N,N′-([(6S)-6-phenylindolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate), which is shown below as Compound II:

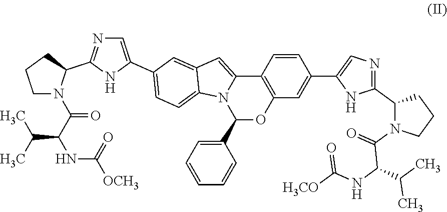 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]

Compound II is described in U.S. Pat. No. 8,871,759.

Compound II, a weak base, has two basic sites, which protonate at low pH giving rise to a sharp pH-dependent solubility profile, particularly between pH 1-3; a normal human stomach has a pH in a range from 1-3 but usually closer to 2. The steep pH-dependent solubility profile has practical implications for dissolution and absorption of Compound II, and likewise for the dissolution and absorption of other weak bases, in the gastrointestinal tract of patients. Specifically, the amount of drug dissolved from formulations of weakly basic compounds in patients with elevated gastric pH could be significantly impaired and more variable, which in turn could lead to potentially lower absorption. See E. Lahner et al., 29 ALIMENTARY PHARMACOL. THER. 1219-1229 (2009); T. L. Russell et al., 11(1) PHARM. RES. 136-143 (1994); G. Krishna et al., 53(3) ANTIMICROB. AGENTS CHEMOTHER. 958-966 (2009).

Elevated gastric pH, or reduced gastric acidity, is known as achlorhydria and can result from a variety of factors. See A. Mitra & F. Kesisoglou, 10 MOL. PHARM. 2970-2979 (2013). Absorption of several drugs such as ketoconazole, itraconazole, atazanavir, cefpodoxime, enoxacin, dipyridamole, nifedipine and digoxin has been shown to be impaired due to this condition. See E. Lahner et al., 29 ALIMENTARY PHARMACOL. THER. 1219-1229 (2009).

Because of the importance of gastric pH in driving dissolution, absorption, and ultimately efficacy of Compound II, it is imperative to develop formulations that can minimize or mitigate the effects of increased gastric pH on Compound II's bioavailability. Such formulations may prove particularly useful in the treatment of HIV patients who are coinfected with HCV. About one-quarter of HIV-infected persons in the United States are also infected with HCV, and these patients tend to have higher gastric pH. See HIV and Viral Hepatitis Fact Sheet, Centers for Disease Control and Prevention (March 2014), available online at http://www.cdc.gov/hepatitis/Populations/PDFs/HIVandHep-FactSheet.pdf.

Solid dispersion formulations have been used previously to promote the oral absorption of poorly water soluble active pharmaceutical ingredients (APIs) (see Ford, 61 PHARM. ACTA HELV. 69-81 (1986)) and to minimize the effect of achlorhydria for weak bases (see M. A. Alam et al., 9(11) EXPERT OPIN. DRUG DELIVERY 1419-1440 (2012); A. Mitra et al., 8 MOL. PHARM. 2216-2223 (2011)). Solid dispersion formulations are compositions in which APIs are dispersed into excipients. Solid solutions, defined as solid dispersions in which the API forms a homogeneous or nearly homogeneous glass when dispersed into the excipient matrix, are of particular interest in the oral delivery of compounds that are poorly water soluble and/or sensitive to gastric pH. Solid dispersion formulations as described above may provide increased absorption of APIs and/or enhanced insensitivity to variations in gastric pH relative to crystalline forms of the API. There remains a need for formulations that provide increased absorption and/or enhanced insensitivity to variations in gastric pH relative to other formulations containing amorphous forms of the API.

The use of solid solution formulations to effectively promote oral drug absorption continues to grow, but their design remains largely a matter of trial and error. There remains a need for solid dispersion formulations of drug substances that may provide effective absorption following oral administration, which is useful to reduce pill burden (e.g., the number of tablets administered), regimen complexity (e.g., eliminating the need to administer with food), and facilitate co-dosing with other medications, such as antacid medications. Formulations with this type of enhanced absorption will ultimately improve compliance, and, therefore, efficacy.

Combining two or more solid dispersion formulations, each containing a drug substance, into a single dosage form may couple the advantages provided by the individual solid dispersions, while providing the additive effect of dosing two or more drug substances. However, designing an effective combination of solid dispersion formulations is dependent on the impact of dosage form characteristics on the properties of the individual solid dispersion formulations.

The current invention relates to novel formulations based on the combination of two solid dispersion formulations, which may provide improved oral absorption, confer insensitivity to higher gastric pH, enhance dissolution rate and/or maintain higher supersaturation of Compound I and Compound II relative to their individual crystalline or amorphous forms.

### SUMMARY OF THE INVENTION

The present disclosure relates to blended compositions comprising (a) a first solid dispersion formulation, which comprises (i) (1aR,5S,8S,10R,22aR)—N-[(1R,2S)-1-[(cyclopropylsulfonamido)carbonyl]-2-ethenylcyclopropyl]-14-methoxy-5-(2-methylpropan-2-yl)-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide hydrate (Compound I):

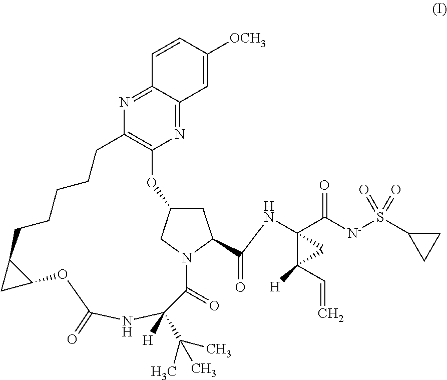 [SMILES: C=C[C@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)[N-]S(=O)(=O)C1CC1]

or a pharmaceutically acceptable salt thereof; (ii) one or more pharmaceutically acceptable polymer or a mixture thereof; and (iii) optionally one or more pharmaceutically acceptable surfactants or a mixture thereof; wherein Compound I and the one or more surfactants, if present, are dispersed in a polymer matrix formed by the one or more pharmaceutically acceptable polymers; (b) a second solid dispersion formulation, which comprises (i) dimethyl N,N′-([(6S)-6-phenylindolo[1,2-c][1,3]benzoxazine-3,10-diyl]bis {1H-imidazole-5,2-diyl-(2S)-pyrrolidine-2,1-diyl[(2S)-3-methyl-1-oxobutane-1,2-diyl]})dicarbamate (Compound II):

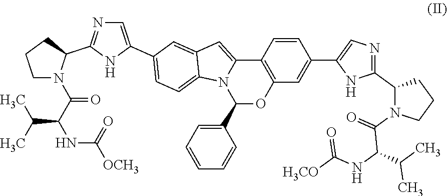 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]

or a pharmaceutically acceptable salt thereof; (ii) one or more pharmaceutically acceptable polymers or a mixture thereof; and (iii) optionally one or more pharmaceutically acceptable surfactants or a mixture thereof; and wherein Compound II and the one or more surfactants, if present, are dispersed in a polymer matrix formed by the one or more pharmaceutically acceptable polymers; and (c) optionally one or more of a diluent, disintegrant, salt, lubricant and glidant. In embodiments, compositions of the disclosure may provide improved oral bioavailability and/or insensitivity to gastric pH.

Other embodiments, aspects and features of the present invention are either further described in or will be apparent from the ensuing description, examples and appended claims.

### BRIEF DESCRIPTION OF THE DRAWINGS

FIG. 1 provides a schematic representation of the formulation process for preparing the solid dispersion intermediate of Compound I as set forth in Example 1.

FIG. 2 provides a schematic representation of the formulation process for preparing Formulation 1 of Example 1.

FIG. 3 provides a schematic representation of the formulation process for preparing the solid dispersion intermediate of Compound I as set forth in Example 2.

FIG. 4 provides a schematic representation of the formulation process for preparing Formulation 2 of Example 2.

FIG. 5 provides a schematic representation of the formulation process for preparing Formulation 3 of Example 3.

FIG. 6 provides a schematic representation of the formulation process for preparing the solid dispersion intermediate of Compound II as set forth in Example 4.

FIG. 7 provides a schematic representation of the formulation process for preparing Formulation 4 of Example 4.

FIG. 8 provides a schematic representation of the formulation process for preparing the granulation intermediate of Compound I as set forth in Example 5.

FIG. 9 provides a schematic representation of the formulation process for preparing the granulation intermediate of Compound II as set forth in Example 5.

FIG. 10 provides a schematic representation of the formulation process for preparing the tablet Formulation 5 of Example 5.

FIG. 11 provides a schematic representation of the formulation process for preparing the tablet Formulation 6 of Example 6.

### DETAILED DESCRIPTION OF THE INVENTION

The instant disclosure is directed to blended compositions comprising two solid dispersion formulations, and optionally one or more excipients. The disclosure is also directed to oral dosage forms, such as tablets or capsules comprising such blended compositions that comprise such solid dispersion formulations.

Compound I is a lipophilic compound (log D˜3 at pH=7) with a low crystallization tendency (Tₘ/T_{g} ratio of 1.12 based on the most stable crystalline phase known, where Tₘ is the melting point of the crystalline form, and T_{g} is the glass transition temperature) and a very low solubility (<7 μg/ml in simulated fasted-state intestinal fluid). Even in its amorphous state, the apparent solubility of neat amorphous Compound I in simulated fasted-state intestinal fluid is 50 μg/mL after two hours of equilibration. In order to enable absorption of Compound I, solid dispersion formulations of Compound I were formulated at a drug loading less than or equal to 40% in combination with absorption-enhancing polymers and surfactants as described in U.S. Provisional Patent Application No. 61/936,019, filed Feb. 5, 2014.

The use of solid dispersion formulations, and, particularly, solid solutions, to promote the oral absorption of poorly water-soluble APIs is known. See, e.g, Ford, 61 PHARM. ACTA. HELV. 69-88 (1986). As discussed above, it is believed that these solid solutions may improve the absorption of orally administered APIs by improving the wetting properties of the API, causing transient supersaturation of the API with respect to a lower energy phase (e.g., crystalline API), or both. In general, solid solutions are believed to enable drug absorption by enhancing the dissolution rate and/or its extent.

Compound II is a weak base, with two basic sites, which protonate at low pH giving rise to pH dependent solubility profile. This pH-dependent solubility could significantly impair the amount of drug dissolved from formulations of weakly basic compounds in patients with elevated gastric pH, which in turn could lead to potentially lower absorption. See E. Lahner et al., 29 ALIMENTARY PHARMACOL. THER. 1219-1229 (2009); T. L. Russell et al., 11(1) PHARM. RES. 136-143 (1994); G. Krishna et al., 53(3) ANTIMICROB. AGENTS CHEMOTHER. 958-966 (2009). In order to mitigate variability in absorption of Compound II due to elevated gastric pH, solid dispersion formulations of Compound II may be formulated at a drug loading less than or equal to 20% in combination with pharmaceutically suitable polymers and surfactants.

Oral dosage forms, combining the solid dispersion formulations of Compound I and of Compound II, may provide fixed-dose combination forms having similar stability and bioavailability for each of Compound I and Compound II as monotherapy formulations.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10%, ±15%, and ±20% and their numerical equivalents.

As used herein, the term “one or more” item includes a single item selected from the list as well as mixtures of two or more items selected from the list.

As used herein, the term “amorphous” indicates that the material lacks a high degree of order on a molecular level and may exhibit the physical properties of a solid or a liquid, depending on the temperature of the material. Amorphous materials do not give distinctive X-ray diffraction patterns.

As used herein, the term “crystalline” indicates that the material has a regular ordered internal structure at the molecular level when in the solid phase, and the crystalline material gives a distinctive X-ray diffraction pattern with defined peaks.

As used herein, the term “substantially amorphous” refers to a composition in which greater than 70%; or greater than 75%; or greater than 80%; or greater than 85%; or greater than 90%; or greater than 95%, or greater than 99% of the compound is amorphous. “Substantially amorphous” can also refer to material that has no more than about 20% crystallinity, or no more than about 10% crystallinity, or no more than about 5% crystallinity, or no more than about 2% crystallinity.

The term “effective amount” indicates a sufficient amount to exert a therapeutic or prophylactic effect. For a patient who is infected with HCV, an effective amount is sufficient to achieve one or more of the following effects: reduce the ability of HCV to replicate, reduce HCV load, and increase viral clearance. For a patient who is not infected with HCV, an effective amount is sufficient to achieve one or more of the following: a reduced susceptibility to HCV infection, and a reduced ability of the infecting virus to establish persistent infection for chronic disease.

The term “subject” (alternatively referred to herein as “patient”) as used herein refers to an animal, preferably a mammal, most preferably a human, who has been the object of treatment, observation or experiment.

Each of Compound I and Compound II, as provided in the solid dispersion formulations, blended compositions and oral dosage forms described herein, independently may take the form of pharmaceutically acceptable salts. The term “pharmaceutically acceptable salt” refers to a salt of the parent compound that has activity and that is not biologically or otherwise undesirable (e.g., is neither toxic nor otherwise deleterious to the recipient thereof); also included in this term are complexes that comprise solvent molecules and a salt of the parent compound. Suitable salts include acid addition salts that may, for example, be formed by mixing a solution of a compound with a solution of a pharmaceutically acceptable acid such as hydrochloric acid, sulfuric acid, acetic acid, trifluoroacetic acid, benzoic acid, phosphoric acid, methanesulfonic acid, naphthalene-1,5-disulfonic acid, and toluenesulfonic acid. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts), and salts formed with suitable organic ligands, such as quaternary ammonium salts. Also, in the case of an acid (—COOH) or alcohol group being present, pharmaceutically acceptable esters can be employed to modify the solubility or hydrolysis characteristics of the compound.

The term “polymer” as used herein refers to a chemical compound or mixture of compounds consisting of repeating structural units created through a process of polymerization. Suitable polymers useful in this invention are described throughout. When specific polymers that are suitable for use in the compositions of the present invention are blended, the blends of such polymers may also be suitable. Thus, the term “polymer” is intended to include blends of polymers in addition to a single species of polymer.

In the embodiments described herein, any variable or component is as defined in the first instance where the variable or component occurs, unless otherwise indicated. When any variable or component occurs more than one time, its selection on each occurrence is independent of its selection at every other occurrence, unless it is expressly stated otherwise. Also, combinations of embodiments, variables or components are permissible only if such combinations result in stable formulations, blends, or oral dosage forms.

### First Solid Dispersion Formulation

A first solid dispersion formulation, also called the first granulation intermediate, comprises (a) Compound I or a pharmaceutically acceptable salt thereof; (b) one or more pharmaceutically acceptable polymers; and (c) optionally one or more pharmaceutically acceptable surfactants.

Compound I, or a pharmaceutically acceptable salt thereof, is present in a concentration of from about 0.1% w/w to about 40% w/w. In particular instances, Compound I, or a pharmaceutically acceptable salt thereof, is present in a concentration of from about 5% w/w to about 35% w/w, or from about 10% w/w to about 30% w/w. All other variables are as provided above.

Compound I may be in the form of a pharmaceutically acceptable salt. In instances, the pharmaceutically acceptable salt of Compound I may be selected from sodium, potassium, calcium, magnesium and quaternary ammonium salts of Compound I. Additional suitable salts include acid addition salt that may, for example, be formed by mixing a solution of a compound with a solution of a pharmaceutically acceptable acid, such as hydrochloric acid, sulfuric acid, acetic acid, trifluoroacetic acid or benzoic acid. In particular instances, the pharmaceutically acceptable salt of Compound I may be a sodium salt or a potassium salt of Compound I. In even more particular instances, the pharmaceutically acceptable salt of Compound I is a potassium salt of Compound I.

The one or more pharmaceutically acceptable polymers may enhance the absorption of the API when used in the solid dispersion formulations described herein. The one or more pharmaceutically acceptable polymers are selected from the group consisting of cellulosic polymers and vinyl pyrrolidone/vinyl acetate copolymers.

Cellulosic polymers include cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate); cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate (HPMCAS)). Commercially available examples of these include hydroxypropyl methylcellulose (HPMC) E3, HPMC E5, HPMC E6, HPMC E15, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, and HPMC P 55.

The pharmaceutically acceptable polymer may be vinyl pyrrolidone/vinyl acetate copolymers. In particular instances, the pharmaceutically acceptable polymer is copovidone, a copolymer of 1-vinyl-2-pyrrolidone and vinyl acetate in the mass proportion of 3:2. Other useful copolymers contain vinyl pyrrolidone and vinyl acetate in ratios of, for example, 90:10, 80:20, 70:30, and 50:50. The amount of vinyl pyrrolidone can range from about 40% up to about 99.9%, and the amount of vinyl acetate can range from about 0.1% up to about 60%. Other vinyl polymers and copolymers having substituents that are hydroxy, alkyl, acyloxy, or cyclic amides include polyethylene polyvinyl alcohol copolymers; and polyvinyl caprolactam-polyvinyl acetate-polyethylene glycol graft copolymer (SOLUPLUS®, BASF Corp.). Commercially available copolymers of vinyl pyrrolidone and vinyl acetate include PLASDONE® S630 (Ashland, Inc., Covonton, Ky.) and KOLLIDON® VA 64 (BASF Corp., Florham Park, N.J.), which contain vinyl pyrrolidone and vinyl acetate in a 60:40 ratio. Other copolymers of vinyl pyrrolidone and vinyl acetate can also be used in the invention. Preferably, the copolymer contains at least 40% vinyl pyrrolidone, although smaller amounts of vinyl pyrrolidone can also be utilized.

The one or more pharmaceutically acceptable polymers are present in a concentration of from about 0.01% w/w to about 90% w/w. In particular instances, the one or more pharmaceutically acceptable polymers are present in a concentration of from about 10% w/w to about 70% w/w, or about 65% w/w. All other variables are as provided above.

The action of polymers may be improved by the presence of one or more pharmaceutically acceptable surfactants. The surfactants can increase the rate of dissolution by facilitating wetting, thereby increasing the maximum concentration of dissolved drug. The surfactants may also make the dispersion easier to process. Surfactants may also stabilize the amorphous dispersions by inhibiting crystallization or precipitation of the drug by interacting with the dissolved drug by such mechanisms as complexation, formation of inclusion complexes, formation of micelles, and adsorption to the surface of the solid drug. Surfactants may also facilitate absorption of APIs by altering API permeability and/or efflux directly. See, e.g., Yu et al., 16 PHARM RES. 1812-1817 (1999). Non-limiting examples of pharmaceutically acceptable surfactants that are suitable for the present invention include polyoxyethylene castor oil derivates, e.g. polyoxyethyleneglycerol triricinoleate or polyoxyl 35 castor oil (CREMOPHOR® EL; BASF Corp.) or polyoxyethyleneglycerol oxystearate such as polyethylenglycol 40 hydrogenated castor oil (CREMOPHOR® RH 40, also known as polyoxyl 40 hydrogenated castor oil or macrogolglycerol hydroxystearate) or polyethylenglycol 60 hydrogenated castor oil (CREMOPHOR® RH 60); or polysorbates or mono fatty acid esters of polyoxyethylene sorbitan, such as a mono fatty acid ester of polyoxyethylene (20) sorbitan, e.g. polyoxyethylene (20) sorbitan monooleate (commercially available as TWEEN® 80), polyoxyethylene (20) sorbitan monostearate (commercially available as TWEEN® 60), polyoxyethylene (20) sorbitan monopalmitate (commercially available as TWEEN® 40), or polyoxyethylene (20) sorbitan monolaurate (commercially available as TWEEN® 20). Other non-limiting examples of suitable surfactants include polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; polyoxyethylene alkylaryl ethers, e.g. polyoxyethylene (2) nonylphenyl ether, polyoxyethylene (3) nonylphenyl ether, polyoxyethylene (4) nonylphenyl ether, polyoxyethylene (3) octylphenyl ether; polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (lauroglycol, such as lauroglycol FCC); sucrose fatty acid esters, e.g. sucrose monostearate, sucrose distearate, sucrose monolaurate, sucrose dilaurate; sorbitan fatty acid mono esters such as sorbitan mono laurate (commercially available as SPAN® 20), sorbitan monooleate, sorbitan monopalnitate (commercially available as SPAN® 40), or sorbitan stearate; D-alpha-tocopheryl polyethylene glycol 1000 succinate (vitamin E TPGS); or a combination or mixture thereof. Other non-limiting examples of suitable surfactants include anionic surfactants, e.g. docusate potassium, docusate sodium, docusate calcium and sodium lauryl sulfate (SLS). Other suitable surfactants include, but are not limited to, block copolymers of ethylene oxide and propylene oxide, also known as polyoxyethylene polyoxypropylene block copolymers or polyoxyethylene polypropyleneglycol, such as POLOXAMER ® 124, POLOXAMER ® 188, POLOXAMER ® 237, POLOXAMER 388, or POLOXAMER 407 (BASF Corp.). As described above, a mixture of surfactants can be used in a solid composition of the present invention. In particular instances, the surfactant is selected from the group consisting of sodium lauryl sulfate (SLS), D-α-tocopheryl polyethylene glycol 1000 succinate (vitamin E TPGS), or nonionic ethoxylated alcohols like polysorbate or poloxamer. In particular instances, the surfactant is selected from sodium lauryl sulfate (SLS) and vitamin E TPGS. All other variables are as provided above.

The one or more pharmaceutically acceptable surfactant may be present in a concentration of from about 2% w/w to about 20% w/w. In particular instances, the one or more pharmaceutically acceptable surfactant is present in a concentration of from about 3% w/w to about 10% w/w, or about 5% w/w. All other variables are as provided above.

The first solid dispersion formulation may be in the form of a particle or may be comprised of particles, with all other variables as provided above.

The first solid dispersion formulation described herein relates to solid dispersion formulations produced by solvent removal (e.g., spray drying), introduction of an antisolvent (e.g., precipitation), addition of heat together with mixing (e.g., extrusion), mechanical activation or other means (e.g., to produce a “solid dispersion intermediate”). That is, the solid dispersion formulation may be formed by a process selected from spray drying and extrusion, such as hot melt extrusion, of the composition. In particular instances, the solid dispersion formulation comprises particles of the composition formed by spray drying.

The first solid dispersion formulation described herein may be prepared by processes that are suitable for causing Compound I to form a dispersion (also referred to as an amorphous dispersion) in the polymer such that the drug is generally amorphous or dissolved in the polymer or a component of the composition, such as a surfactant. The dispersions are stable, and the drug does not form crystals or other insoluble particles. Such methods include solution methods, such as spray drying, spray coating, freeze drying, and evaporation of a co-solvent under vacuum or by heating a solution of polymer and drug. Such methods also include methods that blend the solid drug with the polymer in the molten state, such as hot melt extrusion, and methods of compounding the solid non-molten polymer and drug under heat and pressure to form a dispersion. If the dispersion is effectively a homogeneous molecular dispersion of the individual components, it may also be described as a solid solution, a specific subclass of solid dispersions.

Spray drying is well known (see, e.g., Masters, Spray Drying Handbook, 1991, 5^{th} edition, Longman Scientific & Technical) and widely practiced in a variety of industrial applications including spray drying of milk (see, e.g., U.S. Pat. No. 4,187,617) and pharmaceutical products (see, e.g., U.S. Pat. No. 6,763,607). To produce solid dispersion compositions, by spray drying, the polymer, drug, and optional surfactant, are dissolved in a solvent and then are sprayed through a nozzle as a fine spray into a chamber, where the solvent is evaporated quickly to make particles comprising polymer, drug, and surfactant. Ideally, the solvent is any solvent in which all of the components of the composition are soluble and that is readily evaporated in a spray dryer. The solvent should also be suitable for use in preparing pharmaceutical compositions. In certain embodiments of the invention, the use of mixed-solvent systems, particularly those containing a combination of water and another solvent, are necessary to facilitate the production of solid solution intermediates containing Compound I, an absorption enhancing polymer or polymer(s), and, optionally a surfactant.

Useful solvents for spray drying include water, acetone, ethanol, methanol, dichloromethane, isopropanol and tetrahydrofuran (THF). The spray drying may be performed in a mixed-solvent system. A mixed-solvent system is a solvent system that comprises a first solvent and a second solvent. In aspects, the first solvent may be selected from the group consisting of acetone, ethanol, methanol, dichloromethane, isopropanol and tetrahydrofuran (THF); the second solvent is water. In particular aspects, the first solvent may be selected from the group consisting of ethanol, methanol and acetone; the second solvent is water. In specific instances, the first solvent is acetone and the second solvent is water. The proportions of the first solvent to second solvent may be about 90:10. Mixed-solvent systems are described in International Patent Application Publication No. WO2007/109605 and U.S. Patent Application Publication No. US2007/0026083. Solids loading, which usually refers to the concentration of solid components in the spray-drying solvent system, does not typically exceed 50% and depends on solution properties, such as solubility, stability and viscosity. The solids, comprising Compound I, the pharmaceutically acceptable polymer and any optional surfactant, are present in the spray drying solution in a concentration of from about 5% w/w to about 50% w/w, based on the solubility, stability and viscosity of the solution. In particular instances, the solids are present in the solution in a concentration of from about 10% w/w to about 30% w/w.

Following formation of a solid dispersion formulation, the resulting spray dried intermediate can undergo a secondary drying step to remove residual solvents. This secondary drying unit operation can occur in a static dryer or agitated dryer. Gas, humidified gas, or vacuum may be applied to the material in the secondary dryer and such application can be useful in more rapidly removing residual solvents that remain in the spray-dried intermediate. See, e.g., European Patent Application No. EP1855652 A2 (and references therein) and International Patent Application Publication No. WO2008/012617A1 (and references therein).

In hot melt extrusion, the polymer, drug, and optional surfactant may be either premixed together (e.g., via a wet granulation process) or fed as independent feed streams into the extruder (see Polymer Extrusion 4^{th} Edition by Chris Rauwendaal 2001, Hanser Gardner Publications, Inc., Cincinnati, Ohio or Schenck et al., (2010), Achieving a Hot Melt Extrusion Design Space for the Production of Solid Solutions, in Chemical Engineering in the Pharmaceutical Industry: R&D to Manufacturing (ed. D. J. am Ende), John Wiley & Sons, Inc., Hoboken, N.J., USA). In accordance with this embodiment, any means for preparing a melt in any convenient apparatus in which an admixture of Compound I, a polymer and, optionally a surfactant can be heated and optionally mixed can be used. Solidification can be carried out by cooling the melt. Once a solid is obtained, the solid can be further mechanically processed to provide a convenient form for incorporation into a medicament, for example, tablets or capsules.

It will be appreciated that other methods of preparing a melt, solidifying it, and forming the solid into conveniently sized particles can be utilized without departing from the spirit of the invention. For example, compositions of the invention may be prepared using an extruder. When an extruder is employed to prepare compositions of the invention, the material may be introduced into the extruder either in a pre-flux state, that is, as a dry admixture, or in a fluxed state, that is in a melted, plastic, or semi-solid state achieved after the application of sufficient heat to the admixture to cause Compound I to dissolve in the polymer, optionally when a fluxed charge is prepared, blending may be employed during heating to promote uniformity of the fluxed material.

If the material is introduced to the extruder in a fluxed state, residence time in the extruder is selected to be just sufficient to ensure homogeneity of the composition and the temperature is preferably maintained in the extruder at a level just sufficient to insure that the material maintains its plasticity so that it can be extruded into a conveniently shaped extrudate. If the material is introduced into an extruder in a pre-flux state, the extruder components, for example, the barrels and any mixing chamber present in the equipment, will be maintained at a temperature sufficient to promote fluxing of the admixture. Temperatures selected for use in processing a composition will also take into account that blending occurring within the extruder equipment, for example, in a mixing section of the barrels, will also contribute to localized fluxing of the admixture by imparting shear-stresses that induce heating in the mixture. Additionally, it will be appreciated that equipment temperatures and residence times will be selected to minimize the amount of time that the admixture placed into the extruder spends under conditions of heating and/or shear stress so as to minimize the amount of Compound I, which is decomposed during formation of the composition, as discussed above. In general, extrusion processes in which heating is applied to the material extruded are termed “hot melt extrusion processes.” When compositions of the present invention are prepared using extrusion equipment, the extrudate thus provided can be in any convenient shape, for example, noodles, cylinders, bars, or the like. If desired, the extrudate can be further processed, for example by milling, to provide a particulate form of the composition.

As demonstrated by the Examples, the oral absorption of Compound I when formulated as a solid solution together with a pharmaceutically acceptable polymer, such as copovidone together with surfactants including sodium lauryl sulfate and vitamin E TPGS, is superior to formulations based on undispersed amorphous Compound I.

The relative amount of drug, polymer and optional surfactant can vary widely. The optimal amount of the polymer and optional surfactant can depend, for example, the hydrophilic lipophilic balance (HLB), melting point, and water solubility of the copolymer, and the surface tension of aqueous solutions of the surfactant, etc.

The compositions of the first solid dispersion formulation comprise an effective amount of Compound I, but comprise less than 50% w/w of Compound I due to the poor absorption seen with formulations having greater than 50% w/w of Compound I. Thus, the concentration of Compound I can vary from about 0.1% to about 40.0%, from about 5.0% to about 35.0%, or from about 10% to about 30%, by weight based on the total combined weight of the drug substance polymer, and optional surfactant (not including other excipients).

The concentration of the surfactant can vary from about 2.0% to about 20%, or about 3% to about 10%, or about 5% by weight based on the total combined weight of the drug substance polymer, and optional surfactant (not including other excipients).

The concentration of the pharmaceutically acceptable polymer is added to the concentrations of the Compound I and surfactant to add up to 100%. The concentration can vary from about 0.01% to about 90%, or from about 10% to about 70%, or about 65% by weight based on the total combined weight of the drug substance, polymer and optional surfactant, not including other excipients.

In embodiments, the first solid dispersion formulation may comprise from about 0.1% to about 40% of Compound I or a pharmaceutically acceptable salt thereof, from about 2.0% to about 20% surfactant, with the balance of the formulation being the pharmaceutically acceptable polymer.

Second Solid Dispersion Formulation

A second solid dispersion formulation comprises (a) Compound II or a pharmaceutically acceptable salt thereof; (b) one or more pharmaceutically acceptable polymers; and (c) optionally one or more pharmaceutically acceptable surfactants.

Compound II, or a pharmaceutically acceptable salt thereof, is present in a concentration of from about 5% w/w to about 50% w/w. In particular instances, Compound II, or a pharmaceutically acceptable salt thereof, is present in a concentration of from about 10% w/w to about 40% w/w, or about 20% w/w. All other variables are as provided above.

The one or more pharmaceutically acceptable polymer, which are described above with respect to the first solid dispersion formulation, may be non-ionic.

The one or more pharmaceutically acceptable polymers are selected from the group consisting of cellulosic polymers and vinyl pyrrolidinone/vinyl acetate copolymers, which are provided above with respect to the first solid dispersion formulation. In particular aspects of this embodiment, the one or more pharmaceutically acceptable polymer is selected from the group consisting of hydroxypropylmethyl cellulose (HPMC), hydroxypropylmethyl cellulose acetate succinate (HPMCAS) and hydroyxpropylmethyl cellulose phthalate (HPMCP). In particular instances, the one or more pharmaceutically acceptable polymer is HPMC. All other variables are as provided above.

The one or more pharmaceutically acceptable polymers are present in a concentration of from about 50% w/w to about 95% w/w. In instances, the one or more pharmaceutically acceptable polymers are present in a concentration of from about 50% w/w to about 90% w/w, or about 70% w/w. All other variables are as provided above.

The action of polymers may be improved by the presence of one or more pharmaceutically acceptable surfactants, which are as described above with respect to the first solid dispersion formulation. In aspects of this second solid dispersion formulation, the surfactant may be selected from the group consisting of sodium lauryl sulfate (SLS), D-α-tocopheryl polyethylene glycol 1000 succinate (vitamin E TPGS), or nonionic ethoxylated alcohols like polysorbate or poloxamer. In particular instances, the one or more pharmaceutically acceptable surfactant is vitamin E TPGS. All other variables are as provided above.

The one or more pharmaceutically acceptable surfactant may be present in a concentration of from about 2% w/w to about 20% w/w. In particular instances, the one or more pharmaceutically acceptable surfactant is present in a concentration of from about 5% w/w to about 15% w/w, or about 10% w/w. All other variables are as provided above.

The second solid dispersion formulation may be in the form of a particle or may be comprised of particles, with all other variables as provided above.

The second solid dispersion formulation of Compound II may be formed by a process selected from spray drying and extrusion of the composition, as described above with respect to the first solid dispersion formulation. In particular instances, the second solid dispersion formulation comprises particles of the composition formed by spray drying.

In particular instances, the spray drying is performed in a mixed-solvent system, as described above. Useful solvents for spray drying include water, acetone, ethanol, methanol, dichloromethane, isopropanol and tetrahydrofuran (THF). In aspects, the mixed-solvent system a first solvent and a second solvent, in which the first solvent may be selected from the group consisting of acetone, ethanol, methanol, dichloromethane, isopropanol and tetrahydrofuran (THF); the second solvent is water. In particular aspects, the first solvent may be selected from the group consisting of ethanol, methanol and acetone; the second solvent is water. In specific instances, the first solvent is acetone, and the second solvent is water. The proportions of the first solvent to second solvent may be about 90:10, about 80:20, about 70:30, or about 60:40. Mixed-solvent systems are described in International Patent Application Publication No. WO2007/109605 and U.S. Patent Application Publication No. US2007/0026083. Solids loading, which usually refers to the concentration of solid components in the spray drying solvent system, does not typically exceed 50% and depends on solution properties, such as solubility, stability and viscosity. The solids, comprising Compound II, the pharmaceutically acceptable polymer and surfactant, are present in the spray drying solution in a concentration of from about 5% w/w to about 25% w/w, based on the solubility, stability and viscosity of the solution. In particular instances, the solids are present in the solution in a concentration of from about 10% w/w to about 20% w/w.

As demonstrated by the Examples, the oral absorption of Compound II when formulated as a solid solution together with one or more pharmaceutically acceptable polymer, such as HPMC, together with optional surfactants, such as vitamin E TPGS, is superior to formulations based on undispersed amorphous Compound II.

The relative amount of drug, polymer and optional surfactant can vary widely. The optimal amount of the polymer and optional surfactant can depend, for example, the hydrophilic lipophilic balance (HLB), melting point, and water solubility of the copolymer, and the surface tension of aqueous solutions of the surfactant, etc.

The compositions of the second solid dispersion formulation comprise an effective amount of Compound II, but comprise less than about 50% w/w of Compound II due to the poor absorption seen with formulations having greater than 50% w/w of Compound II. Thus, the concentration of Compound II can vary from about 0.1% to about 40.0%, from about 5.0% to about 35.0%, or from about 10% to about 30%, by weight based on the total combined weight of the drug substance, polymer, and optional surfactant (not including other excipients).

The concentration of the surfactant in the second solid dispersion formulation can vary from about 2.0% to about 20%, or about 5% to about 15%, or about 10% by weight based on the total combined weight of the drug substance, polymer, and surfactant (not including other excipients).

The concentration of the pharmaceutically acceptable polymer in the second solid dispersion formulation is added to the concentrations of the Compound II and surfactant to add up to 100%. The concentration can vary from about 50% to about 95% by weight based on the total combined weight of the drug substance, polymer and optional surfactant, not including other excipients.

In embodiments, the second solid dispersion formulation may comprise from between 5% to 50% of Compound II or a pharmaceutically acceptable salt thereof, 2.0% to about 20% surfactant, with the balance of the formulation being the pharmaceutically acceptable polymer.

### Blended Compositions

Embodiments of the invention relate to blended compositions that comprise the first solid dispersion formulation of Compound I, the second solid dispersion formulation of Compound II, and optionally one or more of a diluent, disintegrant, salt, lubricant and glidant. In all embodiments, all variables with respect to the solid dispersion formulations are as provided above.

In a first embodiment, the first solid dispersion formulation, comprising Compound I as described above, is present in the blended composition in a concentration of from about 3% w/w to about 45% w/w. In particular instances, the first solid dispersion formulation is present in the blended composition in a concentration of from about 13% w/w to about 32% w/w, or about 22% w/w.

In a second embodiment, the second solid dispersion formulation, comprising Compound II, is present in the blended composition in a concentration of from about 6% w/w to about 20% w/w. In particular aspects, the second solid dispersion formulation is present in the blended composition in a concentration of from about 10% w/w to about 18% w/w, or about 16% w/w.

In a third embodiment, the diluent in the blended composition is one or more pharmaceutically acceptable diluents selected from the group consisting of mannitol, microcrystalline cellulose, calcium carbonate, sodium carbonate, lactose, dicalcium phosphate, sodium phosphate and kaolin, and combinations thereof. In particular aspects, the diluent is one or more selected from the group consisting of lactose, microcrystalline cellulose, mannitol and dicalcium phosphate. In a particular instance, the diluent is a combination of lactose, mannitol and microcrystalline cellulose.

In a fourth embodiment, the diluent is present in the blended composition in a concentration of from about 3% w/w to about 58% w/w. In particular instances, the diluent is present in a concentration of from about 18% w/w to about 50% w/w, or about 38% w/w.

In a fifth embodiment, the disintegrant in the blended composition is selected from the group consisting of croscarmellose sodium, sodium starch glycolate and crospovidone. In particular instances, the disintegrant is croscarmellose sodium.

In a sixth embodiment, the disintegrant is present in the blended composition in a concentration of from about 5% w/w to about 20% w/w. In particular instances, the disintegrant is present in a concentration of from about 6% w/w to about 15% w/w, or about 10% w/w.

In a seventh embodiment, the salt is selected from the group consisting of NaCl, KCl, CaCl₂, KH₂PO₄, NaH₂PO₄, K₂SO₄, NaHCO₃, K₂CO₃, and combinations thereof. In aspects, the salt in the blended composition is selected from the group consisting of NaCl, KCl, CaCl₂, and combinations thereof. In a particular instance, the salt is NaCl.

In an eighth embodiment, the salt is present in the blended composition in a concentration of from about 4% w/w to about 30% w/w. In particular instances, the salt is present in a concentration of from about 7% w/w to about 18% w/w, or about 10% w/w.

In a ninth embodiment, the lubricant in the blended composition is selected from the group consisting of magnesium stearate and sodium stearyl fumarate. In a particular instance, the lubricant is magnesium stearate.

In a tenth embodiment, the lubricant is present in the blended composition in a concentration of from about 0.5% w/w to about 3% w/w. In particular instances, the lubricant is present in a concentration of from about 1% w/w to about 2% w/w, or about 1.5% w/w.

In an eleventh embodiment, the glidant in the blended composition is selected from the group consisting of starch, talc, magnesium stearate, and silicon dioxide and combinations thereof. In a particular instance, the glidant is silicon dioxide.

In a twelfth embodiment, the glidant is present in the blended material in a concentration of from about 0% w/w to about 2.5% w/w. In particular instances, the glidant is present in a concentration of from about 0.2% w/w to about 0.9% w/w, or about 0.32% w/w.

A thirteenth embodiment is directed to a process for preparing a blended composition comprising the steps of: a) preparing a first blended material by i) preparing a first solid dispersion formulation comprising Compound I as described above by spray drying, extruding, milling or other known or later-discovered process for making a solid dispersion formulation, ii) blending the first solid dispersion formulation with one or more of a diluent, disintegrant, salt, lubricant and glidant, and iii) optionally granulating to produce the first blended material; b) preparing a second blended material by i) preparing a second solid dispersion formulation comprising Compound II as described above by spray drying, extruding, milling or other known or later-discovered process for making a solid dispersion formulation, ii) blending the second solid dispersion formulation with one or more of a diluent, disintegrant, salt, lubricant and glidant, and iii) optionally granulating to produce the second blended material; c) mixing the first blended material, the second blended material and optionally one or more of a diluent, disintegrant, salt, lubricant and glidant together; and d) optionally granulating the blend of step c) to produce a blended composition. In aspects of this embodiment, blending a first or second solid dispersion formulation with an excipient may comprise blending alone, blending followed by granulation, or granulation followed by blending with the excipients. Similarly, the blend of step c) may be granulated prior to mixing with excipients in step d) or the blend of step c) may be mixed with excipients and co-granulated after mixing. Granulation, as used herein, includes all known and later-developed methods of creating granules.

In aspects of these embodiments, the diluents, disintegrants, salts, lubricants, and/or glidants are as described above. The diluents, disintegrants, salts, lubricants, and/or glidants may be present in the concentrations described above.

Oral Dosage Forms

In a fourteenth embodiment, the blended composition is formulated as a tablet or as a capsule.

A fifteenth embodiment of the invention is directed to a process for preparing a solid pharmaceutical composition comprising the steps of: a) preparing a blended composition as described above in the thirteenth embodiment; b) compressing the blended composition into a tablet or filling into a capsule. In aspects of the fifteenth embodiment, the tablet is optionally film-coated; in further aspects, the tablet or capsule is optionally photo-shielded, for example by use of a blister packaging.

A sixteenth embodiment of the invention is directed to a process for preparing a solid pharmaceutical composition comprising the steps of: a) preparing a blended composition as described above in the thirteenth embodiment; b) compressing the blended composition into a tablet or filling into a capsule. In aspects of the sixteenth embodiment, the tablet is optionally film-coated; in further aspects, the tablet or capsule is optionally photo-shielded, for example by use of a blister packaging.

In aspects of these embodiments, the diluents, disintegrants, salts, lubricants and/or glidants are as described above with respect to blended compositions. The diluents, disintegrants, salts, lubricants and/or glidants may be present in the concentrations described above with respect to blended compositions.

Pharmaceutical compositions intended for oral use may be prepared from the solid dispersion formulations and blended materials described above in accordance with the methods described herein and other methods known to the art for the manufacture of pharmaceutical compositions. Such compositions may further contain one or more agents selected from the group consisting of sweetening agents, flavoring agents, coloring agents and preserving agents in order to provide pharmaceutically elegant and palatable preparations.

Tablets may contain the active ingredient in admixture with non-toxic pharmaceutically acceptable excipients that are suitable for the manufacture of tablets. These excipients may be for example, inert diluents, granulating and disintegrating agents, binding agents, glidants, lubricating agents, and antioxidants, for example, propyl gallate, butylated hydroxyanisole and butylated hydroxy toluene. The tablets may be uncoated or they may be coated to delay disintegration and absorption in the gastrointestinal tract and thereby provide a sustained action over a longer period.

Compositions for oral use may also be presented as capsules (e.g., hard gelatin) wherein the active ingredient is mixed with an inert solid diluent, for example, calcium carbonate, calcium phosphate or kaolin, or as soft gelatin capsules wherein the active ingredient is mixed with liquids or semisolids, for example, peanut oil, liquid paraffin, fractionated glycerides, surfactants or olive oil. Aqueous suspensions contain the active materials in mixture with excipients suitable for the manufacture of aqueous suspensions. Dispersible powders and granules suitable for preparation of an aqueous suspension by the addition of water provide the active ingredient in mixture with a dispersing or wetting agent, suspending agent and one or more preservatives. In certain embodiments of the invention, the pharmaceutical compositions of the invention include a diluent system, lubricant, glidant and filmcoat, at concentrations of from about 3% w/w to about 58% w/w, from about 0.5% w/w to about 3% w/w, from about 0% w/w to about 3.0% w/w, and from about 1% w/w to about 5% w/w respectively, or at from about 13% w/w to about 32% w/w, from about 1.0% w/w to about 2.0%, from about 0.2% w/w to about 0.9% w/w and from about 2.0% w/w to about 4.0% w/w, respectively. In certain embodiments, the solid dispersion formulations are blended with a diluent, one or more disintegrating agents, lubricant and glidant. An exemplary blended composition or oral dosage form includes mannitol, croscarmellose sodium, sodium chloride, colloidal silica and magnesium stearate.

The disintegrant may be present in a concentration from about 5% w/w to about 20% w/w or from about 6% w/w to about 15% w/w. A salt may be also present, which may be sodium chloride, potassium chloride or a combination thereof. The combination of salts and disintegrant is present at a concentration from about 12% w/w to about 35% w/w of the final pharmaceutical composition. Pharmaceutical compositions comprising these levels of disintegrant and salt (in combination with polymer(s)) provides a rapidly disintegrating dosage form. Rapidly disintegrating tablets based on solid dispersion formulations are disclosed in U.S. Pat. No. 7,189,415.

The blended compositions may be roller compacted or wet granulated to densify and/or reduce the risk of segregation of components during subsequent handling (e.g., compression into tablets). Granulation steps can also be used to minimize the impact of raw material property variability (e.g., excipient particle size) on subsequent processing (e.g., tablet compression) and ultimate product performance. Lubrication is typically performed prior to roller compaction and tablet compression to reduce the tendency of material to adhere to compression surfaces (e.g., tablet tooling). In particular embodiments, the lubricant is magnesium stearate. These methods can be carried out by those skilled in the art. See, e.g., Ansel, Introduction to Pharmaceutical Dosage Forms, Seventh Edition, 1999.

To prepare the pharmaceutical compositions of the invention, the solid dispersion formulation or blended composition is compressed into an oral dosage form including tablets or capsules. Tablets can be prepared with a variety of possible shapes (ellipsoidal, capsule, biconvex round, etc.). The powder can also be encapsulated in capsule dosage (e.g., using hard gelatin capsules). Techniques suitable for preparing solid oral dosage forms of the present invention are described in Remington's Pharmaceutical Sciences, 18th edition, edited by A. R. Gennaro, 1990, Chapter 89 and in Remington—The Science and Practice of Pharmacy, 21st edition, 2005, Chapter 45. In certain embodiments, the first solid dispersion formulation is present in an amount of from about 3% w/w to about 45% w/w of the pharmaceutical composition or from about 12% w/w to about 32% w/w of the final pharmaceutical composition, and the second solid dispersion formulation is present in an amount of from about 8% w/w to about 25% w/w of the pharmaceutical composition or from about 11% w/w to about 21% w/w of the final pharmaceutical composition.

As demonstrated by the examples, a solid dispersion formulation of Compound II showed robust pharmacokinetic performance when dosed with pH-raising medication, relative to the conventional formulation. When the combination formulation containing the solid dispersion formulation of Compound I and the solid dispersion formulation of Compound II was prepared as an oral dosage form as described herein, it was found to maintain the pharmacokinetic performance of each of Compound I and Compound II, and to provide robust absorption regardless of gastric pH modulation due to the use of H2-receptor antagonists and proton-pump inhibitors.

Additional Combination Dosage Forms

Additional embodiments include combination regimens, comprising the fixed dose combinations as described above and one or more additional drug substance(s). For combination regimens, other drug substance(s) can be added to the solid solution or the tablet formulation, either in a crystalline form, neat amorphous form, or as a solid solution. In particular combination regimens, one or more additional drug substance(s) are formulated into one or more solid dispersion formulations, and the solid dispersion formulation of Compound I, the solid dispersion formulation of Compound II and the solid dispersion formulation(s) of the additional drug substance(s) are combined into a blended composition and provided as a dosage form that may be a tablet or capsule. Additional components may also be combined into the blended composition, such as diluents, disintegrants, salts, lubricants and glidants, as described above.

Exemplary drug substances that may be included as the additional drug substance(s) include, but are not limited to, HCV protease inhibitors, HCV polymerase inhibitors, HCV NS4A inhibitors, HCV NS5A inhibitors, and HCV NS5b inhibitors.

HCV protease inhibitors include, but are not limited to, those disclosed in U.S. Pat. Nos. 8,080,654; 7,973,040; 8,828,930; 8,927,569; 7,879,797; 7,470,664; 8,216,999; 8,377,873; 8,278,322; 8,138,164; 8,377,874; 8,309,540; 8,591,878; 7,494,988; 7,485,625; 7,795,250; 7,449,447; 7,442,695; 7,425,576; 7,342,041; 7,253,160; 7,244,721; 7,205,330; 7,192,957; 7,186,747; 7,173,057; 7,169,760; 7,012,066; 6,914,122; 6,911,428; 6,894,072; 6,846,802; 6,838,475; 6,800,434; 6,767,991; 5,017,380; 4,933,443; 4,812,561 and 4,634,697; U.S. Patent Application Publication Nos. US2014/0057836, US2013/0178413, US2010/0099695, US2014/0296136, US2002/0068702, US2002/0160962, US2005/0119168, US2005/0176648, US2005/0209164, US2005/0249702 and US2007/0042968; and International Patent Application Publication Nos. WO2014/025736, WO2009/010804, WO2010/011566, WO2011/014487, WO2006/119061, WO2007/015855, WO2007/015787, WO2007/016441, WO2007/131966, WO2007/148135, WO2008/057209, WO2008/051475, WO2008/057208, WO2008/051514, WO2009/108507, WO2008/051477, WO2012/040040, Wo2013/074386, WO03/006490, WO03/087092, WO04/092161 and WO08/124148.

HCV protease inhibitors also include, but are not limited to, boceprevir, narlaprevir, vaniprevir, grazoprevir, VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott), TMC-435350 (Medivir), ITMN-191/R7227 (InterMune/Roche), EA-058 (Abbott/Enanta), EA-063 (Abbott/Enanta), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhibitors include, but are not limited to, those disclosed in Landro et al., 36(31) BIOCHEMISTRY 9340-9348 (1997); Ingallinella et al., 37(25) BIOCHEMISTRY 8906-8914 (1998); Llinás-Brunet et al., 8(13) BIOORG. MED. CHEM. LETT. 1713-1718 (1998); Martin et al., 37(33) BIOCHEMISTRY 11459-11468 (1998); Dimasi et al., 71(10) J. VIROL. 7461-7469 (1997); Martin et al., 10(5) PROTEIN ENG. 607-614 (1997); Elzouki et al., 27(1) J. HEPAT. 42-48 (1997); 9(217) BIOWORLD TODAY 4 (Nov. 10, 1998); U.S. Patent Application Publication Nos. US2005/0249702 and US 2007/0274951; and International Patent Application Publication Nos. WO98/14181, WO98/17679, WO98/22496, WO99/07734 and WO05/087731.

HCV polymerase inhibitors include, but are not limited to, those disclosed in U.S. Pat. No. 8,183,216; U.S. Patent Application Publication Nos. US2011/0306573, US2014/0206640 and US2014/0161770; and International Patent Application Publication Nos. WO09/040269, WO2013/177219, WO2014/058801, WO2014/062596, and WO2012/142085.

HCV polymerase inhibitors include, but are not limited to, VP-19744 (Wyeth/ViroPharma), PSI-7851 (Pharmasset), GS-7977 (sofosbuvir, Gilead), R7128 (Roche/Pharmasset), PF-868554/filibuvir (Pfizer), VCH-759 (ViroChem Pharma), HCV-796 (Wyeth/ViroPharma), IDX-184 (Idenix), IDX-375 (Idenix), NM-283 (Idenix/Novartis), R-1626 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH222 (ViroChem), VCH916 (ViroChem), VCH716(ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., 7(4) CURRENT OPINION IN DRUG DISCOVERY AND DEVELOPMENT 446 (2004); Tan et al., 1 NATURE REVIEWS 867 (2002); and Beaulieu et al., 5 CURRENT OPINION IN INVESTIGATIONAL DRUGS 838 (2004).

HCV NS4A inhibitors include, but are not limited to, those disclosed in U.S. Pat. Nos. 7,476,686 and 7,273,885; U.S. Patent Application Publication No. US2009/0022688; and International Patent Application Publication Nos. WO2006/019831 and WO2006/019832. Additional HCV NS4A inhibitors include, but are not limited to, AZD2836 (Astra Zeneca) and ACH-806 (Achillon Pharmaceuticals, New Haven, Conn.).

HCV NS5A inhibitors include, but are not limited to, those disclosed in U.S. Pat. Nos. 8,871,759 and 8,609,635; U.S. Patent Application Publication No. US2014/0371138; and International Patent Application Publication Nos. WO2014/110705 and WO2014/110706.

HCV NS5B inhibitors include, but are not limited to, those disclosed in U.S. Patent Application Publication No. US2012/0328569; and International Patent Application Publication Nos. WO2010/111483, WO2011/106992, WO2011/106985 and WO2011/106929.

A further embodiment of the invention is directed to a process for preparing a solid pharmaceutical composition comprising the steps of: a) preparing a first blended material by i) preparing a first solid dispersion formulation comprising Compound I as described above by spray drying, extruding, milling or other known or later-discovered process for making a solid dispersion formulation, ii) blending the first solid dispersion formulation with one or more of a diluent, disintegrant, salt, lubricant and glidant, and iii) granulating, such as by roller compaction to produce the first blended material; b) preparing a second blended material by i) preparing a second solid dispersion formulation comprising Compound II as described above by spray drying, extruding, milling or other known or later-discovered process for making a solid dispersion formulation, ii) blending the second solid dispersion formulation with one or more of a diluent, disintegrant, salt, lubricant and glidant, and iii) granulating to produce the second blended material; d) preparing one or more blended material(s) by i) preparing one or more additional solid dispersion formulation(s) comprising one or more additional APIs by spray drying, extruding, milling or other known or later-discovered process for making a solid dispersion formulation, ii) blending the one or more additional solid dispersion formulation with one or more of a diluent, disintegrant, salt, lubricant and glidant, and iii) granulating, such as by roller-compaction to produce the one or more additional blended material; d) mixing the first blended material, the second blended material and the one or more additional blended material together; e) mixing the blend of step d) with a lubricant and optionally one or more of a diluent, disintegrant, salt and glidant together; and f) optionally granulating the blend of step e) to produce a blended composition; g) compressing the particles into a tablet or filling into a capsule. In addition, the tablet is optionally film-coated; in further aspects, the tablet or capsule is optionally photo-shielded, for example by use of a blister packaging.

The following examples serve only to illustrate the invention and its practice. The examples are not to be construed as limitations on the scope or spirit of the invention.

In addition, the following abbreviations are used throughout this specification and in the Examples. Each of these terms has the meaning listed below.

### ABBREVIATIONS

AUC_{0-∞} Area under the concentration time curve from time zero to infinity
bar Metric unit of pressure, 1 bar=100,000 Pascal
CI Confidence interval
Cₘₐₓ Maximum concentration (specifically of a drug)
g Gram(s)
GM Geometric mean
GMR Geometric mean ratio
HPMC Hydroxypropylmethyl cellulose
hr Hour(s)
kg Kilogram(s)
kp, kpf Kilopond, a non-standard gravitational unit of force, also kilogram-force; 1 kp=9.80665 Newtons
L Liter
mg Milligram
min Minute(s)
mL Milliliter
mm Millimeter
nM Nanomolar
PSI, psi Pounds per square inch [gauge], 1 Pascal=0.000145037738007 psi
RPM Revolutions per minute
SLS Sodium lauryl sulfate
TPGS Vitamin E polyethylene glycol succinate
w/w, % w/w Percentage by weight (i.e., grams of solute in 100 g of solution)
μM Micromolar

### EXAMPLES

### Example 1

### Copovidone Sodium Laurel Sulfate Formulation of Compound I

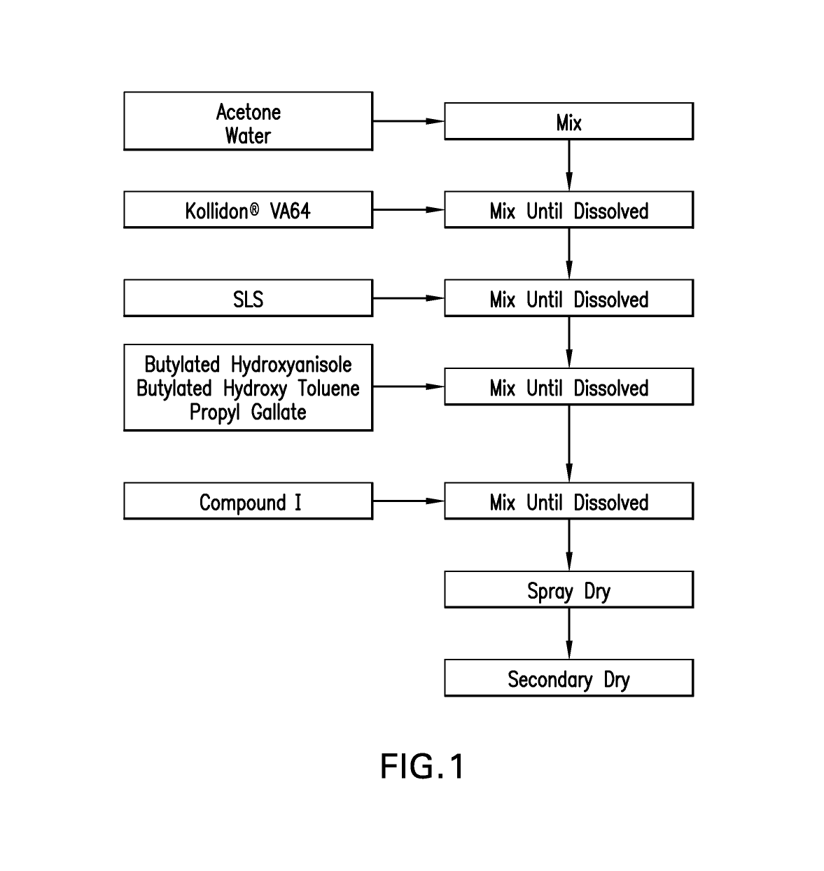

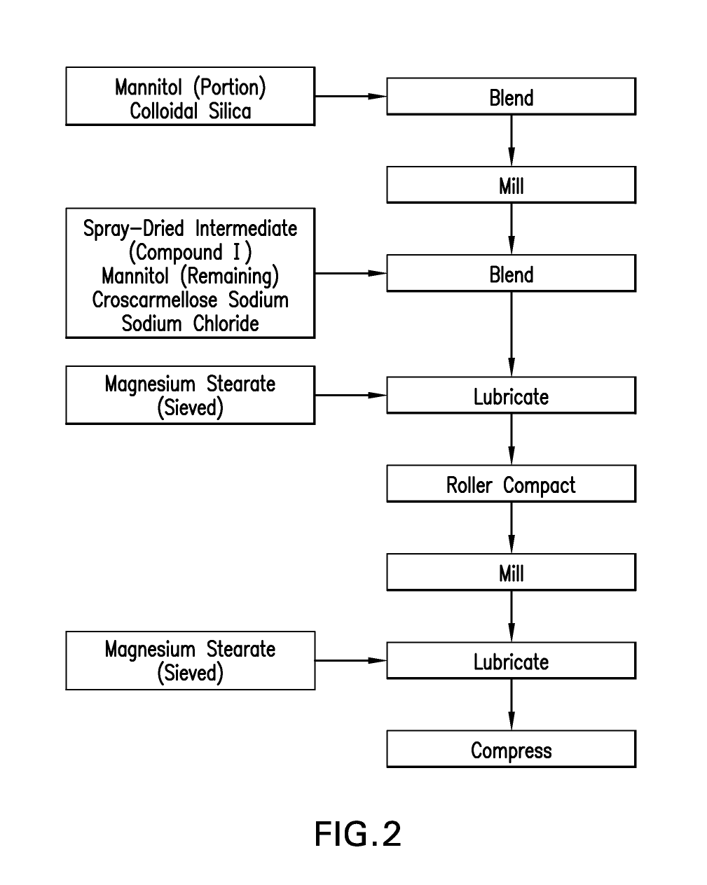

Formulation 1 is a tablet composition (Table 1) based on a spray-dried intermediate of Compound I dispersed into copovidone and SLS. FIG. 1 illustrates the process for preparing the spray-dried intermediate, and FIG. 2 illustrates the process used to produce Formulation 1. To produce Formulation 1, Compound I, copovidone, and SLS are dissolved into a 90/10 (w/w) acetone/water solution. This spray-drying solution is prepared such that it contains 20% w/w solids in solution. The spray-drying solution is then pumped through a spray drying nozzle (e.g., a pressure nozzle) in order to produce a plume of atomized droplets. These droplets are dried in a chamber that can contain an inert heated gas (e.g., nitrogen). The particles thus produced are collected (e.g., using a cyclone). Typically, a secondary-drying operation is used to sufficiently dry the spray-dried intermediate. Humid nitrogen or air may be used to facilitate drying. Tray dryers or agitated dryers can be used to perform this secondary-drying operation. The dried spray-dried intermediate is added to the “downstream tablet” components listed in Table 1, except the magnesium stearate, where the colloidal silica and a portion of mannitol are co-screened with a QUADRO COMILL equipped with a round impeller and 32 R screen, processed at 2000 RPM, and the remaining components may be screened through a No. 30 mesh and blended using a 600-L BOHLE BLENDER for 21 minutes at 6 RPM. One-third of the magnesium stearate (screen through No. 60 mesh) is added to the blender, and the mixture is lubricated for 6 minutes at 10 RPM. The blend was then granulated into ribbons using an ALEXANDERWERK WP 120 ROLLER COMPACTOR with a 40 mm knurled roll operating at a roll pressure of 29-39 bar with a roll gap of 2.0 mm. The ribbons were subsequently milled using the rotary fine granulator equipped with 2.0 mm and 1.0 mm size CONIDUR® screens. The granules were then lubricated with the remaining magnesium stearate (screened through No. 60 mesh) in the 60 L BOHLE blender for 6 minutes at 10 RPM. The lubricated granules were then compressed on a rotary tablet press to a 1000 mg image tablet using 16-24 tablet stations with size 7.94 mm×19.05 mm caplet tooling. The hardness of the tablets was measured to be between 15 and 25 kiloponds (kp=1 kgf).

TABLE 1
Composition of Formulation 1

| | |
|---|---|
| Component | Amount (mg/tablet) |

Spray-Dried Intermediate

| | |
|---|---|
| Compound I | 100.0 |
| Polyvinylpyrolidone/Vinyl Acetate | 214.2 |
| Copolymer (Kollidon VA64) | |
| Butylated Hydroxyanisole | 0.8333 |
| Butylated Hydroxy Toluene | 0.8333 |
| Propyl Gallate | 0.8333 |
| Sodium Lauryl Sulfate | 16.67 |

Blended Material & Downstream Tablet

| | |
|---|---|
| Mannitol | 449.2 |
| Croscarmellose Sodium | 100.0 |
| Sodium Chloride | 100.0 |
| Colloidal Silicon Dioxide | 2.500 |
| Magnesium Stearate (non-bovine) | 15.00 |
| Total | 1000 |

### Example 2

### Copovidone-TPGS Formulation of Compound I

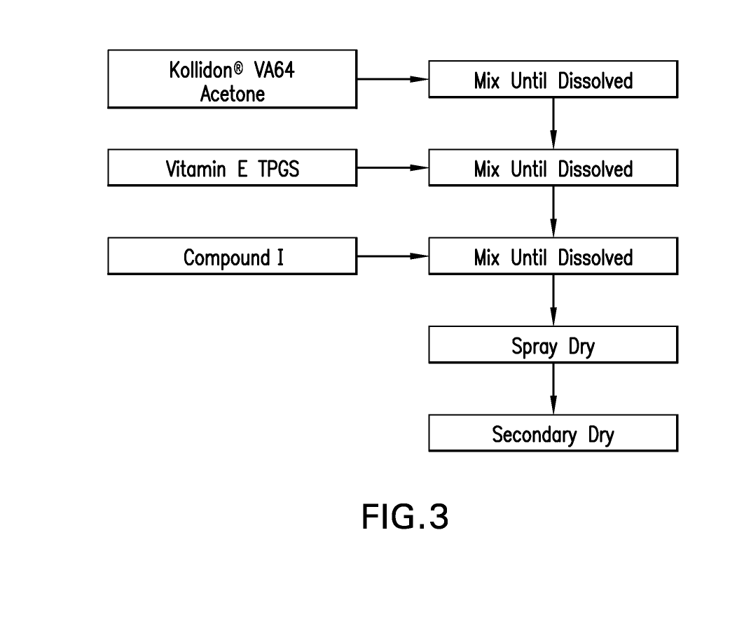

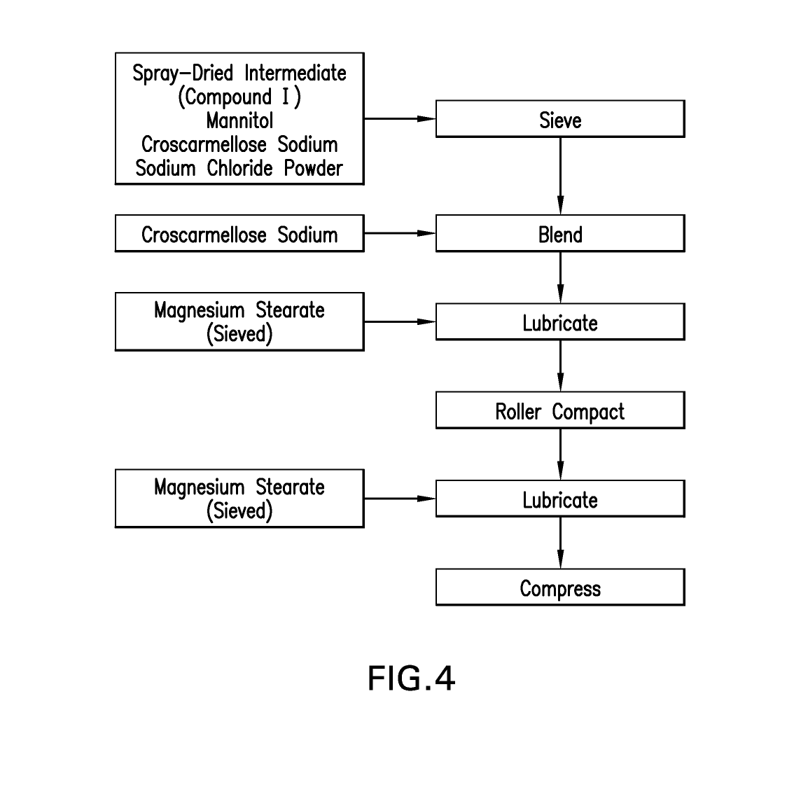

A formulation (Formulation 2) of Compound I, Vitamin E TPGS, and copovidone was prepared as described by Table 2, using acetone as the spray drying solvent. FIG. 3 illustrates the process for preparing the spray-dried intermediate, and FIG. 4 illustrates the process used to produce Formulation 2. The concentration of Compound I in the dry spray-dried intermediate of Formulation 2 was 30% w/w in comparison with the 30% w/w spray-dried intermediate used in Formulation 1. The three solid components of the spray drying solution were incorporated into the solution at 20% w/w. A NIRO PSD-1 spray dryer with a pressure nozzle was used to produce the spray-dried particles. Heated nitrogen was supplied to the spray dryer at an inlet temperature sufficient to maintain a 30° C. outlet temperature and a gas flow rate of 1850 g/min. The spray drying solution flow rate was 140-170 g/min, which required a nozzle pressure of approximately 200-400 PSI.

The dried spray-dried intermediate (screen through No. 30 mesh) is blended with the “downstream tablet” components listed in Table 3 (screen through No. 30 mesh, except croscarmellose sodium), except the magnesium stearate, using a rotary blender for 10 minutes at 25 RPM. One-third of the magnesium stearate (screen through No. 60 mesh) is added to the blender, and the mixture is lubricated for 5 minutes at 25 RPM. The blend was then granulated into ribbons using an ALEXANDERWERK WP 120 ROLLER COMPACTOR with a 25 mm knurled roll operating at a roll pressure of 19 bar with a roll gap of 2.0 mm. The ribbons were subsequently milled using the rotary fine granulator equipped with 2.0 mm and 1.0 mm size CONIDUR® screens. The granules were then blended with the remaining magnesium stearate (screened through No. 60 mesh) in the rotary blender for 5 minutes at 25 RPM. The lubricated granules were then compressed on a rotary tablet press (PICCOLA) to a 1000 mg image tablet using 2 tablet stations with size 9.74 mm×19.05 mm caplet tooling. The hardness of the tablets was measured to be between 14 to 24 kiloponds (kp=1 kgf).

TABLE 2
Composition of Formulation 2

| | |
|---|---|
| Components | Amount (mg/tablet) |

Spray-Dried Intermediate

| | |
|---|---|
| Compound I | 100.0 |
| Polyvinylpyrolidone/Vinyl Acetate | 216.7.0 |
| Copolymer (KOLLIDON ® VA64) | |
| Vitamin E TPGS | 16.67 |

Blended Material & Downstream Tablet

| | |
|---|---|
| Mannitol | 449.2 |
| Croscarmellose Sodium | 100.0 |
| Sodium Chloride | 100.0 |
| Colloidal Silicon Dioxide | 2.500 |
| Magnesium Stearate (non-bovine) | 15.00 |
| Total | 1000 |

TABLE 3
Pharmacokinetic Data for Compound I Following Oral
Administration to Male Beagle Dogs (Fasted State;
100 mg of Compound I; Pentagastrin Was Administered
Intramuscularly at a Target Dose Level of 6
μg/kg, 0.05 mL/kg 30 ± 5 Minutes Prior to Dosing)

| | | | Formulation 2/ |
|---|---|---|---|
| Parameters | Formulation 1 | Formulation 2 | Formulation 1 |
| AUC₀₋₂₄ₕᵣ | 80.7 ± 16.9 | 87.4 ± 10.7 | 1.08 |
| (μM*hr) | | | |
| Cₘₐₓ (μM) | 11.6 ± 1.99 | 11.5 ± 0.742 | 1.01 |

This example illustrates successful absorption of Compound I in an animal model from an orally administered formulation based on a solid solution intermediate comprising Compound I, copovidone, and vitamin E TPGS.

### Example 3

### Conventional Roller Compacted Formulation of Compound II

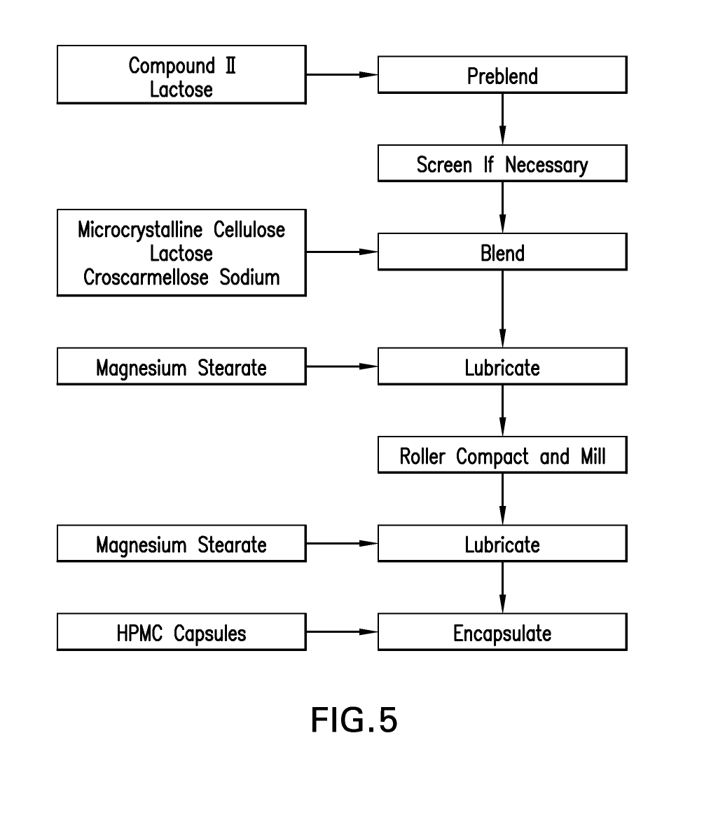

The conventional roller compacted formulation of Compound II is given in Table 4. An amorphous form of Compound II, as described in U.S. Pat. No. 8,871,759, in particular Example 223, was roller compacted and filled into capsules to produce Formulation 3. FIG. 5 outlines the process used to make Formulation 3.

TABLE 4
Composition of Formulation 3

| Components | mg/capsule |
|---|---|
| Compound II | 10.00 |
| Microcrystalline Cellulose | 81.40 |
| Lactose Monohydrate | 81.40 |
| Croscarmellose Sodium | 5.400 |
| Magnesium Stearate (non-bovine) (intra-granular) | 0.9000 |
| Magnesium Stearate (non-bovine) (extra-granular) | 0.9000 |
| Capsule fill weight | 180.0 |
| White Opaque HPMC Capsule | 48.00 |
| Total Capsule Weight | 228.0 |

The oral absorption obtained from Formulation 3 with and without pH-modifying medications (20 mg famotidine) was evaluated as part of human clinical study. For the treatment with famotidine, single oral doses of 20 mg famotidine (1×20 mg tablet) were administered on the previous evening and 2 hours prior to a single oral dose of Formulation 3. The results are shown in Table 5.

TABLE 5
Comparison of Human Pharmacokinetics of Formulation 3 With and Without
20 mg Famotidine (100 mg Dose; Healthy Subjects)

| | | | Formulation 3 + | | Formulation 3 + |
|---|---|---|---|---|---|
| | Formulation 3 | | famotidine | | famotidine/Formulation 3 |
| Parameters | N | [GM and 95% CI] | N | [GM and 95% CI] | [GMR and 90% CI] |
| AUC_{0-∞} | 10 | 3.77 | 10 | 1.57 | 0.42 |
| (μM · hr) | | [2.90, 4.90] | | [1.25, 1.99] | [0.33, 0.53] |
| Cₘₐₓ (nM) | 10 | 216 | 10 | 76.2 | 0.35 |
| | | [150, 310] | | [54.6, 106] | [0.27, 0.47] |

This example illustrates poor absorption obtained using a conventional formulation of Compound II instead of a solid solution-based formulation upon co-administration with a pH-raising medication.

### Example 4

### HPMC-TPGS Formulation of Compound II

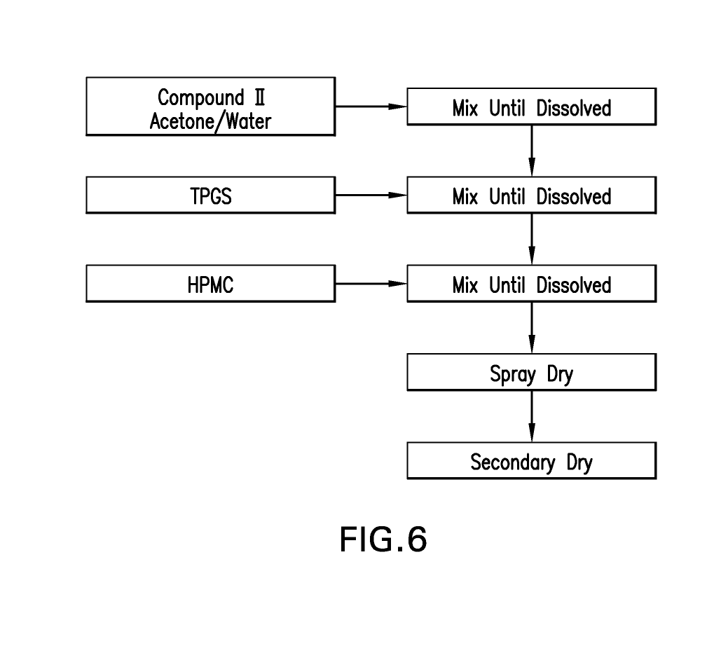

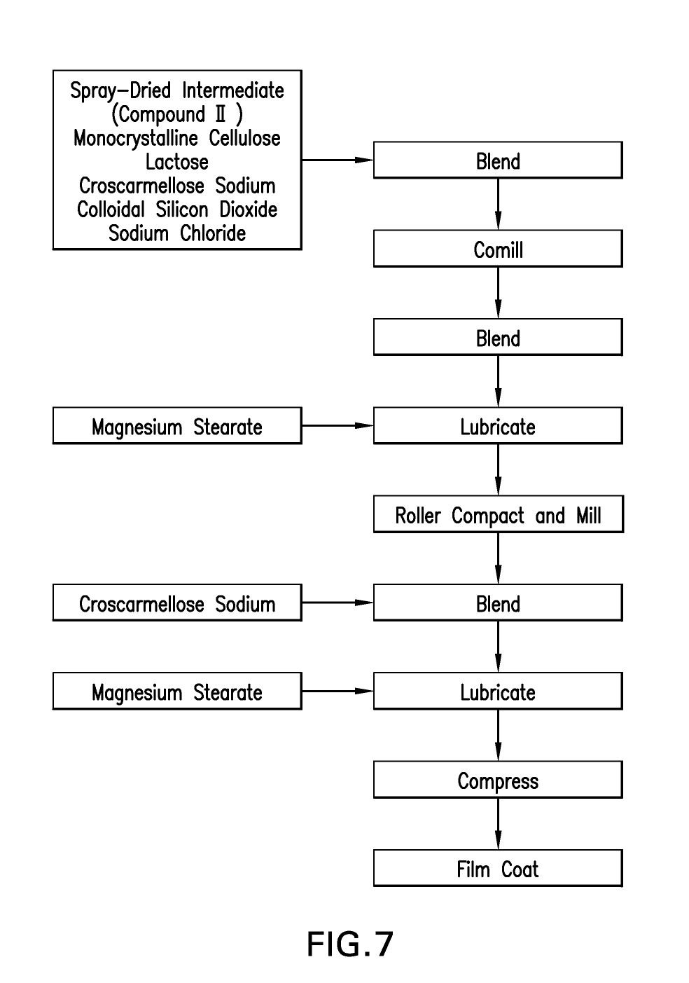

Formulation 4 is a tablet composition containing a solid dispersion formulation of Compound II as shown in Table 6. FIG. 6 illustrates the process for preparing the spray-dried intermediate, and FIG. 7 illustrates the process used to produce Formulation 4. The solid dispersion was prepared from a solid solution comprising Compound II, TPGS, and HPMC, by spray drying from an acetone/water solvent system, as shown in FIG. 4.

TABLE 6
Composition of the Tablet Formulation 4

| | |
|---|---|
| Components | mg/tablet |

Spray-Dried Intermediate

| | |
|---|---|
| Compound II | 50.00 |
| Hypromellose 2910 | 175.0 |
| Vitamin E Polyethylene Glycol Succinate | 25.00 |

Blended Material & Downstream Tablet

| | |
|---|---|
| Cellulose, Microcrystalline | 71.25 |
| Lactose Monohydrate | 71.25 |
| Croscarmellose Sodium | 82.00 |
| Sodium Chloride | 50.00 |
| Silicon Dioxide, Colloidal | 2.500 |
| Magnesium Stearate | 5.000 |
| Film coat | 16.00 |
| Total Tablet Weight | 548.0 |

A NIRO PSD-2 spray dryer with a pressure nozzle was used to produce the spray-dried particles. The spray-dried particles are dried in a chamber that can contain an inert heated gas (e.g., nitrogen). The particles thus produced are collected (e.g., using a cyclone). Typically, a secondary-drying operation is used to sufficiently dry the spray dried intermediate. Humid nitrogen or air may be used to facilitate drying. Tray dryers or agitated dryers can be used to perform this secondary-drying operation.

Heated nitrogen was supplied to the spray dryer at an inlet temperature sufficient to maintain a 50° C. outlet temperature and a gas flow rate of 1856 g/min. The spray drying solution flow rate was 7.14 kg/hr, which required a nozzle pressure of approximately 221 psi using a STEINEN A75 nozzle.

The spray-dried intermediate was blended and roller compacted along with microcrystalline cellulose, lactose, 50 mg of croscarmellose sodium, sodium chloride, colloidal silicon dioxide, and magnesium stearate. Only half of the magnesium stearate was added prior to roller compaction. The second half of the magnesium stearate and the remaining croscarmellose sodium was added after roller compaction, and the resulting powder mix was blended further. A rotary tablet press was used to produce tablets of 50 mg potency.

The impact of pH-raising medication (famotidine) on the co-dosing regimen of 100 mg Compound I (Formulation 1) and 50 mg Compound II (Formulation 4) was studied clinically on healthy subjects. For the treatment with famotidine, single oral doses of 20 mg famotidine (1×20 mg tablet) was administered 10 hours and 2 hours prior to an oral dose of Formulation 1 and Formulation 4. The results as shown in Table 7 indicate that the exposure of Compound II following the administration of famotidine, a pH-raising medication is comparable to that without famotidine.

TABLE 7
Comparison of Human Pharmacokinetics of Formulation 4
With and Without Famotidine, When Co-Dosed with Formulation 1.
(100 mg Compound I/50 mg Compound II)

| | Treatment C: | | | | |
|---|---|---|---|---|---|
| | Formulation 1 + | | Treatment B: | | |
| | Formulation 4 + | | Formulation 1 + | | Treatment C/ |
| | Famotidine | | Formulation 4 | | Treatment B |
| | | GM | | GM | GMR |
| Parameters | N | [95% CI] | N | [95% CI] | [90% CI] |
| AUC_{0-∞} | 12 | 1793.117 | 12 | 1973.325 | 0.91 |
| (nM · hr) | | [1398, 2299] | | [1557, 2501] | [0.74, 1.12] |
| Cₘₐₓ (nM) | 12 | 95.535 | 12 | 102.481 | 0.93 |
| | | [74.4, 123] | | [83.7, 125] | [0.74, 1.17] |

### Example 5

### Fixed-Dose Combination of Compound I and Compound II—Formulation 5

The fixed-dose combination tablet Formulation 5 was obtained by combining the granulation intermediate of Compound I, exactly as described in Example 1 (also Example 2 of

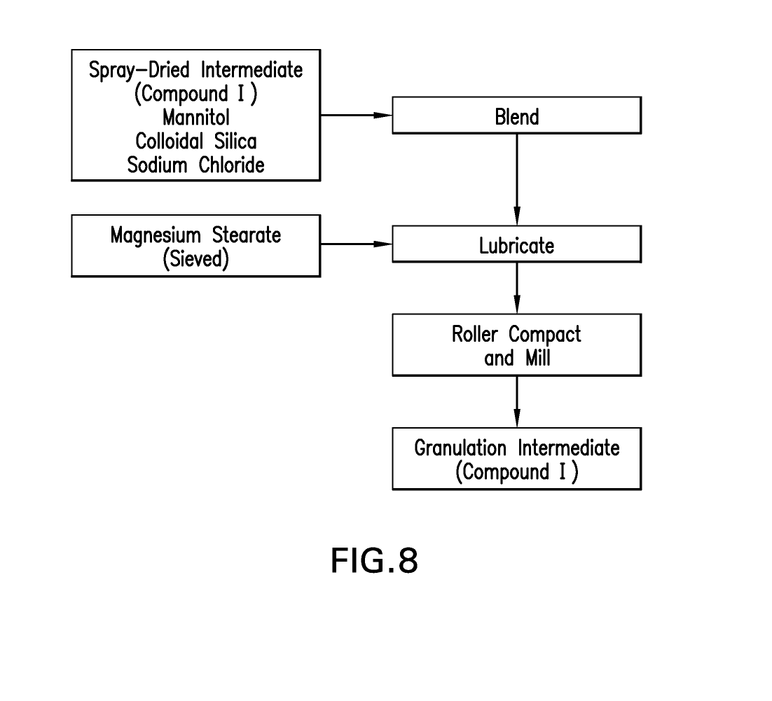

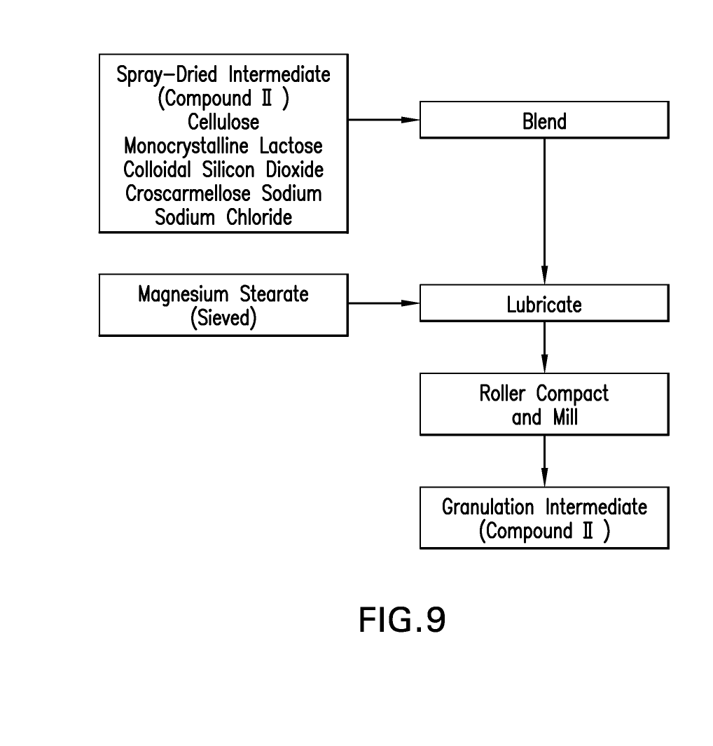

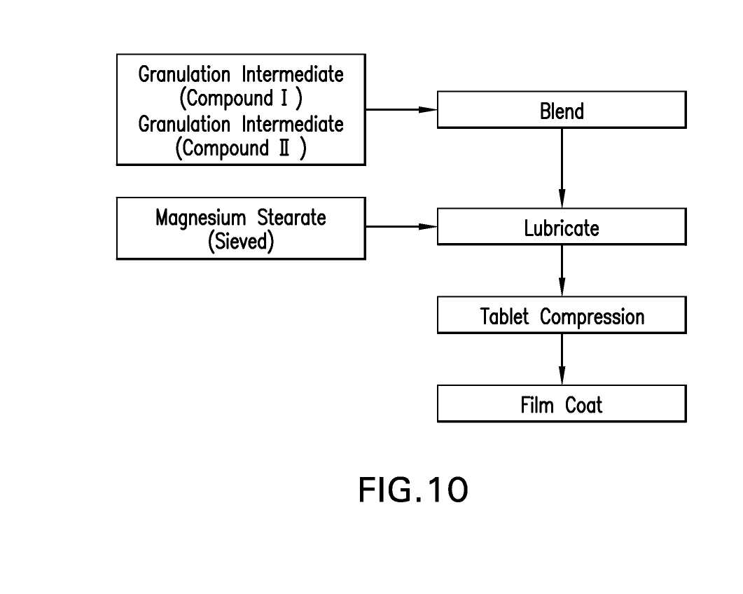

U.S. Provisional Patent Application No. 61/936,019, filed Feb. 5, 2014), with the granulation intermediate of Compound II, as described in Example 4. FIG. 8 illustrates the process for preparing the granulation intermediate of Compound I, and FIG. 9 illustrates the process used to produce the granulation intermediate of Compound II. The resulting blend was lubricated with magnesium stearate and compressed into tablets. FIG. 10 illustrates the process used to produce Formulation 5. A rotary press was used to produce tablets with 100 mg potency of Compound I and 50 mg potency of Compound II. The tablets were film-coated and waxed in a film-coating pan. The composition of the fixed dose combination tablets is shown in Table 8.

TABLE 8
Composition of Fixed-Dose Combination Tablet Formulation 5
(100 mg Compound I/50 mg Compound II)

| Components | mg/tablet |
|---|---|
| Compound I Granulation Intermediate | |
| Compound I Spray-Dried Intermediate | |
| Compound I | 100.0 |
| Sodium Lauryl Sulfate | 16.67 |
| Polyvinylpyrolidone/Vinyl Acetate Copolymer | 216.6 |
| Mannitol SD100 | 449.2 |
| Croscarmellose Sodium | 100.0 |
| Sodium Chloride Powder | 100.0 |
| Silicon Dioxide, Colloidal | 2.500 |
| Magnesium Stearate | 5.000 |
| Compound II Granulation Intermediate | |
| Compound II Spray-Dried Intermediate | |
| Compound II | 50.00 |
| Hypromellose 2910 | 175.0 |
| Vitamin E Polyethylene Glycol Succinate | 25.00 |
| Cellulose, Microcrystalline | 71.24 |
| Lactose Monohydrate | 71.24 |
| Croscarmellose Sodium | 50.00 |
| Sodium Chloride Powder | 50.00 |
| Silicon Dioxide, Colloidal | 2.500 |
| Magnesium Stearate | 2.500 |
| Magnesium Stearate | 15.02 |
| Film Coat | 45.08 |
| Total Tablet Weight | 1547.6 |

The pharmacokinetics of the fixed-dose combination Formulation 5 (100 mg potency of Compound I and 50 mg potency of Compound II), was compared with the co-administration of Formulation 1 (Compound I, 100 mg potency) and Formulation 4 (Compound II, 50 mg potency). These comparisons are summarized in Tables 9 and 10.

TABLE 9
Comparison of Human Pharmacokinetics for Compound I (100 mg) in
Formulation 5, Relative to Co-Administration (100 mg dose, Healthy Subjects)

| | | | | | Formulation 5/ |
|---|---|---|---|---|---|
| | | | Formulation 1 + | | Formulation 1 + |
| | Formulation 5 | | Formulation 4 | | Formulation 4 |
| | N | GM [95% CI] | N | GM [95% CI] | GMR [90% CI] |
| AUC_{0-∞} | 16* | 366 [312,460] | 39* | 411 [350,488] | 0.917 [0.805,1.04] |
| (nM · hr) | | | | | |
| Cₘₐₓ (nM) | 25 | 23.0 [17.9,29.1] | 49 | 24.6 [20.0,29.7] | 0.935 [0.781,1.11] |

*AUC_{0-∞} values excluded for subjects with AUC_{0-∞} that were extrapolated ≧30%

TABLE 10
Comparison of Human Pharmacokinetics for Compound II (50 mg) in
Formulation 5, Relative to Co-Administration
(50 mg dose, Healthy Subjects)

| | | | | | Formulation 5/ |
|---|---|---|---|---|---|
| | | | Formulation 1 + | | Formulation 1 + |
| | Formulation 5 | | Formulation 4 | | Formulation 4 |
| | N | GM [95% CI] | N | GM [95% CI] | GM [95% CI] |
| AUC_{0-∞} | 25 | 2199 [1941,2490] | 49 | 1909 [1677,2198] | 1.14 [1.03,1.26] |
| (nM · hr) | | | | | |
| Cₘₐₓ (nM) | 25 | 125 [111,140] | 49 | 105 [95.4,117] | 1.18 [1.04,1.33] |

This results show that the pharmacokinetic response of the fixed-dose combination Formulation 5 is statistically similar (within the 90% CI) to the co-administration of the Formulation 1 and Formulation 4.

The pharmacokinetics of the Formulation 5 with and without pH raising medication (famotidine or pantoprazole) was also studied. For the treatment with famotidine, single oral doses of 20 mg famotidine (1×20 mg tablet) was administered 10 hours and two hours prior to a single oral dose of fixed-dose combination; Tables 11 and 12 summarize these results. For the treatment with pantoprazole, multiple oral doses of 40 mg of pantoprazole (1×40 mg tablet) were administered QD on days 1 through 5, and a single oral dose of the fixed-dose combination of Formulation 5 was administered 2 hours after pantoprazole dosing on Day 5. The results relating to pantoprazole are shown in Tables 13 and 14. These results show that the pharmacokinetic response of the fixed-dose combination Formulation 5 is statistically similar (within the 90% CI) with or without pH-raising medication.

TABLE 11
Comparison of Human Pharmacokinetics for Compound I (100 mg) in
Formulation 5 With and Without Famotidine (20 mg)

| | | | | | Formulation 5 + |
|---|---|---|---|---|---|
| | | | Formulation 5 + | | Famotidine/ |
| | Formulation 5 | | Famotidine | | Formulation 5 |
| | N | GM [95% CI] | N | GM [95% CI] | GM [95% CI] |
| AUC_{0-∞} | 16 | 0.573 (0.465, .707) | 14 | 0.633 (0.545, 0.735) | 1.10 (0.95, 1.28) |
| (μM · hr) | | | | | |
| Cₘₐₓ (nM) | 16 | 39.4 (28.8, 54.0) | 14 | 35.0 (28.6, 42.8) | 0.89 (0.71, 1.11) |

TABLE 12
Comparison of Human Pharmacokinetics for Compound II (50 mg) in
Formulation 5 With and Without Famotidine (20 mg)

| | | | | | Formulation 5 + |
|---|---|---|---|---|---|
| | | | Formulation 5 + | | Famotidine/ |
| | Formulation 5 | | Famotidine | | Formulation 5 |
| | N | GM [95% CI] | N | GM [95% CI] | GM [95% CI] |
| AUC_{0-∞} | 16 | 2.75 (2.38, 3.19) | 14 | 2.88 (2.44, 3.41) | 1.05 (0.92, 1.18) |
| (uM · hr) | | | | | |
| Cₘₐₓ (μM) | 16 | 0.140 (0.124,0.158) | 14 | 0.156 (0.131, 0.185) | 1.11 (0.98, 1.26) |

TABLE 13
Comparison of Human Pharmacokinetics for Compound I (100 mg) in
Formulation 5 With and Without Pantoprazole (40 mg)

| | | | | | Formulation 5 + |
|---|---|---|---|---|---|
| | | | Formulation 5 + | | Pantoprazole/ |
| | Formulation 5 | | Pantoprazole | | Formulation 5 |
| | N | GM [95% CI] | N | GM [95% CI] | GM [95% CI] |
| AUC_{0-∞} (μM · hr) | 16 | 0.573 (0.465, .707) | 12 | 0.640 (0.534, 0.767) | 1.12 (0.96, 1.30) |
| Cₘₐₓ (nM) | 16 | 39.4 (28.8, 54.0) | 12 | 43.5 (32.7, 57.9) | 1.10 (0.89, 1.37) |

TABLE 14
Comparison of Human Pharmacokinetics for Compound II (50 mg) in
Formulation 5 With and Without Pantoprazole (40 mg)

| | | | | | Formulation 5 + |
|---|---|---|---|---|---|
| | | | Formulation 5 + | | Pantoprazole/ |
| | Formulation 5 | | Pantoprazole | | Formulation 5 |
| | N | GM [95% CI] | N | GM [95% CI] | GM [95% CI] |
| AUC_{0-∞} | 16 | 2.75 (2.38, 3.19) | 12 | 2.88 (2.42, 3.42) | 1.05 (0.93, 1.18) |
| (μM · hr) | | | | | |
| Cₘₐₓ (μM) | 16 | 0.140 (0.124,0.158) | 12 | 0.143 (0.122, 0.169) | 1.02 (0.92, 1.14) |

### Example 6

### Fixed-Dose Combination Tablet Formulation 6

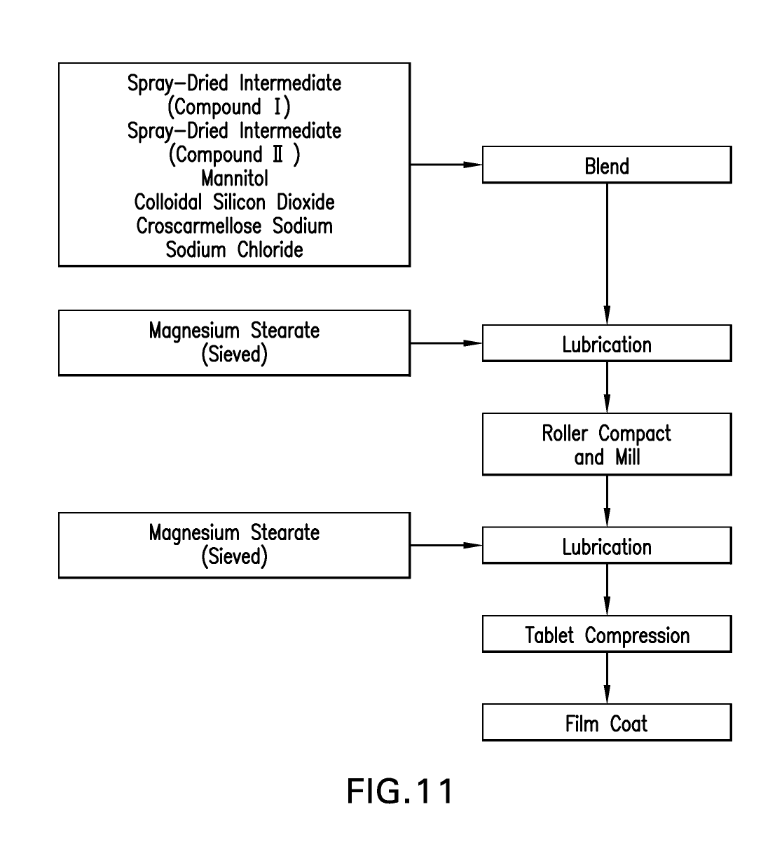

The fixed-dose combination Formulation 6 is prepared by blending the solid dispersion formulations of Compound I (prepared according to Example 1), Compound II (prepared according to Example 4), mannitol, croscarmellose sodium, sodium chloride, silicon dioxide, and magnesium stearate together and roller compacting the blend. The roller-compacted granulation intermediate is then lubricated with magnesium stearate, tableted and film-coated. The tablet composition is provided in Table 15. FIG. 11 illustrates the process for preparing Formulation 6.

TABLE 15
Composition of Fixed Dose Combination of Formulation 6

| | |
|---|---|
| Components | mg/tablet |

Compound I Spray-Dried Intermediate

| | |
|---|---|
| Compound I | 100.0 |
| Sodium Lauryl Sulfate | 16.67 |
| Polyvinylpyrolidone/Vinyl Acetate Copolymer | 216.6 |

Compound II Spray-Dried Intermediate

| | |
|---|---|
| Compound II | 50.00 |
| Hypromellose 2910 | 175.0 |
| Vitamin E Polyethylene Glycol Succinate | 25.00 |

Blended Material and Downstream Tablet

| | |
|---|---|
| Mannitol | 591.68 |
| Croscarmellose Sodium | 150.00 |
| Sodium Chloride Powder | 150.00 |
| Silicon Dioxide, Colloidal | 5.000 |
| Magnesium Stearate | 22.52 |
| Film Coat | 45.08 |
| Total Tablet Weight | 1547.6 |

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art, which are also intended to be encompassed by the following claims.

1. A blended composition comprising
(a) a first solid dispersion formulation, which comprises
(i) (1aR,5S,8S,10R,22aR)—N-[(1R,2S)-1-[(cyclopropylsulfonamido)carbonyl]-2-ethenylcyclopropyl]-14-methoxy-5-(2-methylpropan-2-yl)-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide hydrate (Compound I): 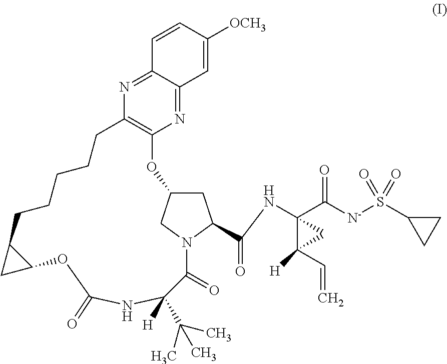 [SMILES: C=C[C@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)[N-]S(=O)(=O)C1CC1]
or a pharmaceutically acceptable salt thereof;
(ii) one or more pharmaceutically acceptable polymers or a mixture thereof; and
(iii) optionally one or more pharmaceutically acceptable surfactants or a mixture thereof;
wherein Compound I and the one or more surfactants, if present, are dispersed in a polymer matrix formed by the one or more pharmaceutically acceptable polymers;
(b) a second solid dispersion formulation, which comprises
(i) dimethyl N,N′-([(6S)-6-phenylindolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate (Compound II): 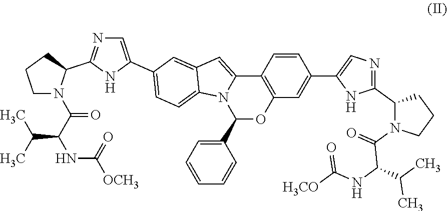 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]
or a pharmaceutically acceptable salt thereof;
(ii) one or more pharmaceutically acceptable polymers or a mixture thereof; and
(iii) optionally one or more pharmaceutically acceptable surfactants or a mixture thereof; and
wherein Compound II and the one or more surfactants, if present, are dispersed in a polymer matrix formed by the one or more pharmaceutically acceptable polymers; and
(c) optionally one or more of a diluent, disintegrant, salt, lubricant and glidant.

2. The blended composition according to claim 1, wherein Compound I and Compound II are substantially amorphous.

3. The blended composition according to claim 1, wherein
a) in the first solid dispersion formulation,
i) Compound I is present in a concentration of from about 0.1% w/w to about 40% w/w, relative to the total combined weight of the first solid dispersion formulation,
ii) the one or more pharmaceutically acceptable polymers or a mixture thereof is present in a concentration of from about 0.01% w/w to about 90% w/w, relative to the total combined weight of the first solid dispersion formulation, and
iii) the one or more pharmaceutically acceptable surfactants is present in a concentration of from about 2% w/w to about 20% w/w, relative to the total combined weight of the first solid dispersion formulation; and
b) in the second solid dispersion formulation,
i) Compound II is present in a concentration of from about 5% w/w to about 50% w/w, relative to the total combined weight of the second solid dispersion formulation,
ii) the one or more pharmaceutically acceptable polymers or a mixture thereof is present in a concentration of from about 50% w/w to about 95% w/w, relative to the total combined weight of the second solid dispersion formulation, and
iii) the one or more pharmaceutically acceptable surfactants is present in a concentration of from about 2% w/w to about 20% w/w, relative to the total combined weight of the second solid dispersion formulation.

4. The blended composition according to claim 3, wherein
a) in the first solid dispersion formulation,
i) Compound I is present in a concentration of from about 5% w/w to about 35% w/w, relative to the total combined weight of the first solid dispersion formulation,
ii) the one or more pharmaceutically acceptable polymers or a mixture thereof is present in a concentration of from about 10% w/w to about 70% w/w, relative to the total combined weight of the first solid dispersion formulation, and
iii) the one or more pharmaceutically acceptable surfactants is present in a concentration of from about 3% w/w to about 10% w/w, relative to the total combined weight of the first solid dispersion formulation; and
b) in the second solid dispersion formulation,
i) Compound II is present in a concentration of from about 10% w/w to about 40% w/w, relative to the total combined weight of the second solid dispersion formulation,
ii) the one or more pharmaceutically acceptable polymers or a mixture thereof is present in a concentration of from about 50% w/w to about 90% w/w, relative to the total combined weight of the second solid dispersion formulation, and
iii) the one or more pharmaceutically acceptable surfactants is present in a concentration of from about 5% w/w to about 15% w/w, relative to the total combined weight of the second solid dispersion formulation.

5. The blended composition according to claim 4, wherein
a) in the first solid dispersion formulation,
i) Compound I is present in a concentration of from about 10% w/w to about 30% w/w, relative to the total combined weight of the first solid dispersion formulation,
ii) the one or more pharmaceutically acceptable polymers or a mixture thereof is present in a concentration of about 65% w/w, relative to the total combined weight of the first solid dispersion formulation, and
iii) the one or more pharmaceutically acceptable surfactants is present in a concentration of about 5% w/w, relative to the total combined weight of the first solid dispersion formulation; and
b) in the second solid dispersion formulation,
i) Compound II is present in a concentration of about 20% w/w, relative to the total combined weight of the second solid dispersion formulation,
ii) the one or more pharmaceutically acceptable polymers or a mixture thereof is present in a concentration of about 70% w/w, relative to the total combined weight of the second solid dispersion formulation, and
iii) the one or more pharmaceutically acceptable surfactants is present in a concentration of about 10% w/w, relative to the total combined weight of the second solid dispersion formulation.

6. The blended composition according to claim 1, wherein
a) in the first solid dispersion formulation, the one or more pharmaceutically acceptable polymers or a mixture thereof is selected from the group consisting of cellulosic polymers and vinyl pyrrolidininone/vinyl acetate copolymers, and mixtures thereof; and
b) in the second solid dispersion formulation, the one or more pharmaceutically acceptable polymers or a mixture thereof is selected from the group consisting of cellulosic polymers.

7. The blended composition according to claim 6, wherein
a) in the first solid dispersion formulation, the one or more pharmaceutically acceptable polymers or a mixture thereof is copovidone; and
b) in the second solid dispersion formulation, the one or more pharmaceutically acceptable polymers or a mixture thereof is HPMC.

8. The blended composition according to claim 1, wherein
a) in the first solid dispersion formulation, the one or more pharmaceutically acceptable surfactant or a mixture thereof is present and is selected from sodium lauryl sulfate and vitamin E TPGS and mixtures thereof; and
b) in the second solid dispersion formulation, the one or more pharmaceutically acceptable surfactant is present and is vitamin E TPGS.

9. The blended composition according to claim 1, further comprising one or more excipient selected from the group consisting of diluents, granulating agents, disintegrants, lubricants, glidants, sweetening agents, flavoring agents, coloring agents, preserving agents, binding agents, and antioxidants.

10. An oral dosage form comprising the blended composition according to claim 1.

11. The oral dosage form according to claim 10, wherein the oral dosage form is a tablet or a capsule.

12. The oral dosage form according to claim 11, wherein the oral dosage form is a tablet, and wherein the tablet is film-coated.

13. A process for preparing a blended composition, comprising
1) preparing a first blended material by
a) preparing a first solid dispersion formulation by spray drying, extruding or milling to form particles, said first solid dispersion formulation comprising
(i) (1aR,5S,8S,10R,22aR)—N—[(R1R,2S)-1-[(cyclopropylsulfonamido)carbonyl]-2-ethenylcyclopropyl]-14-methoxy-5-(2-methylpropan-2-yl)-3,6-dioxo-1,1a,3,4, 5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide hydrate (Compound I): 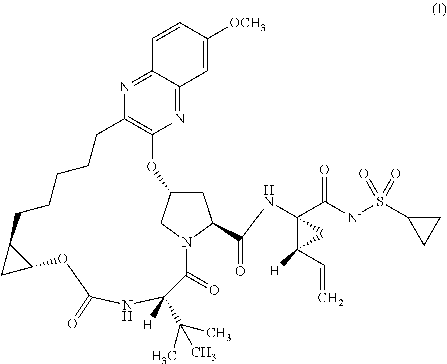 [SMILES: C=C[C@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)[N-]S(=O)(=O)C1CC1]
or a pharmaceutically acceptable salt thereof,
(ii) one or more pharmaceutically acceptable polymers or a mixture thereof, and
(iii) optionally one or more pharmaceutically acceptable surfactants or a mixture thereof,
wherein Compound I and the one or more surfactants are dispersed in a polymer matrix formed by the one or more pharmaceutically acceptable polymers;
b) optionally blending the first solid dispersion formulation with one or more of a diluent, disintegrant, salt, lubricant and glidant, and optionally granulating to form a first blended material;
2) preparing a second blended material by
a) preparing a second solid dispersion formulation by spray drying, extruding or milling to form particles, said second solid dispersion formulation comprising
(i) dimethyl N,N′-([(6S)-6-phenylindolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate (Compound II): 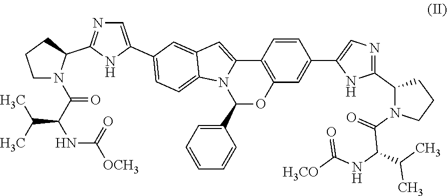 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]
or a pharmaceutically acceptable salt thereof;
(ii) one or more pharmaceutically acceptable polymers or a mixture thereof; and
(iii) optionally one or more pharmaceutically acceptable surfactants or a mixture thereof; and
wherein Compound II and the one or more surfactants are dispersed in a polymer matrix formed by the one or more pharmaceutically acceptable polymers; and
b) optionally blending the second solid dispersion formulation with one or more of a diluent, disintegrant, salt, lubricant and glidant, and optionally granulating to form a second blended material;
3) blending the first blended material, the second blended material, and optionally one or more of a diluent, disintegrant, salt, lubricant and glidant to provide a blended composition.

14. A process for preparing an oral dosage form, comprising
1) preparing a first blended material by
a) preparing a first solid dispersion formulation by spray drying, extruding or milling to form particles, said first solid dispersion formulation comprising
(i) (1aR,5S,8S,10R,22aR)—N—[(R1R,2S)-1-[(cyclopropylsulfonamido)carbonyl]-2-ethenylcyclopropyl]-14-methoxy-5-(2-methylpropan-2-yl)-3,6-dioxo-1,1a,3,4, 5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide hydrate (Compound I): 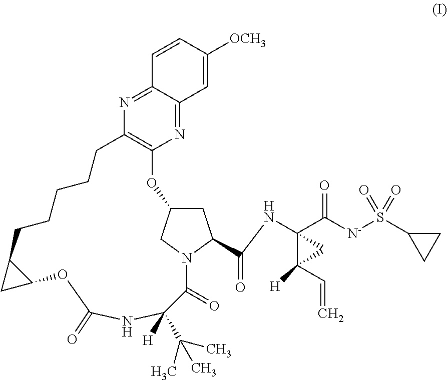 [SMILES: C=C[C@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)[N-]S(=O)(=O)C1CC1]
or a pharmaceutically acceptable salt thereof,
(ii) one or more pharmaceutically acceptable polymers or a mixture thereof, and
(iii) optionally one or more pharmaceutically acceptable surfactants or a mixture thereof,
wherein Compound I and the one or more surfactants are dispersed in a polymer matrix formed by the one or more pharmaceutically acceptable polymers;
b) optionally blending the first solid dispersion formulation with one or more of a diluent, disintegrant, salt, lubricant and glidant, and optionally granulating to form a first blended material;
2) preparing a second blended material by
a) preparing a second solid dispersion formulation by spray drying, extruding or milling to form particles, said second solid dispersion formulation comprising
(i) dimethyl N,N′-([(6S)-6-phenylindolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate (Compound II): 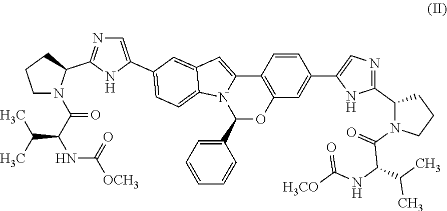 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]
or a pharmaceutically acceptable salt thereof;
(ii) one or more pharmaceutically acceptable polymers or a mixture thereof; and
(iii) optionally one or more pharmaceutically acceptable surfactants or a mixture thereof; and
wherein Compound II and the one or more surfactants are dispersed in a polymer matrix formed by the one or more pharmaceutically acceptable polymers; and
b) optionally blending the second solid dispersion formulation with one or more of a diluent, disintegrant, salt, lubricant and glidant, and optionally granulating to form a second blended material;
3) blending the first blended material, the second blended material, and optionally one or more of a diluent, disintegrant, salt, lubricant and glidant and optionally granulating to provide a blended composition;
4) compressing the particles into a tablet or filling into a capsule.

15. The process according to claim 14, further comprising film-coating the tablet.
